(19) United States
(12) Patent Application Publication
(10) Pub. No.: US 2013/0345115 A1
(43) Pub. Date: Dec. 26, 2013

(54) NUCLEAR PENETRATING H4 TAIL PEPTIDES FOR THE TREATMENT OF DISEASES MEDIATED BY IMPAIRED OR LOSS OF P53 FUNCTION

(71) Applicant: Woojin An, Los Angeles, CA (US)

(72) Inventor: Woojin An, Los Angeles, CA (US)

(73) Assignee: University of Southern California, Los Angeles, CA (US)

(21) Appl. No.: 13/923,243

(22) Filed: Jun. 20, 2013

Related U.S. Application Data
(60) Provisional application No. 61/663,488, filed on Jun. 22, 2012.

(51) Int. Cl. C07K 14/00; A61K 38/16; A61K 45/06

(52) U.S. Cl. CPC C07K 14/00; A61K 45/06; A61K 38/16
USPC 514/1.2; 514/19.3; 514/21.3; 530/324; 536/23.1; 514/44 R; 530/387.9; 536/23.53; 435/375; 435/320.1; 435/325; 435/69.1; 435/18

(57) ABSTRACT

Methods for using chimeric polypeptides and other compositions comprising H4 tail peptides to increase p53 activity in cells and treat diseases mediated by lack of or dysfunctional p53 function as described. Also provided are chimeric polypeptides and other compositions comprising H4 tail peptides.

### CROSS-REFERENCE TO RELATED APPLICATIONS

This application claims the benefit under 35 U.S.C. §119(e) of U.S. Provisional Application No. 61/663,488, filed Jun. 22, 2012, the content of which is hereby incorporated by reference in its entirety.

### STATEMENT OF GOVERNMENT SUPPORT

This invention was made with government support under Grant No. R01 GM084209 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on May 23, 2013, is named 064189-4203_SL.txt and is 8,361 bytes in size.

### FIELD

The present invention relates generally to compositions and methods for treating diseases caused or by impaired or loss of functional p53.

### BACKGROUND

Throughout and within this application various technical and patent literature are referenced either explicitly or by reference to an Arabic numeral. The bibliographic citations for the Arabic numeral citations are found after the experimental examples. The contents of these technical and patent citations are incorporated by reference into this application to more fully describe the state of the art to which this invention pertains.

The p53 tumor suppressor is a key component of cellular mechanisms that respond to different forms of stress, including DNA damage and oncogene activation (1, 2). p53 regulates these processes mainly, if not solely, by functioning as a sequence-specific transcription factor that stimulates expression of a number of target genes embedded within chromatin (3, 4). p53 transcriptional activity in the context of chromatin can require the recruitment and function of chromatin remodeling machineries (5-8). Acetylation of lysine residues on the amino terminal tails of core histones occur during p53-dependent transcription in response to DNA damage (5, 6, 8). When cells are exposed to stress conditions, p53 recruits histone acetyltransferase (HAT) to establish distinct histone acetylation at its target genes, which will in turn allow the transcriptional machinery to access the promoters and initiate the high level of transcription from target genes (5, 6, 8). Histone acetylation is a reversible epigenetic signal, which is dynamically governed by HAT and histone deacetylase (HDAC). For the initiation of p53-mediated DNA damage response, the cellular activity of HAT dominates over HDAC activity to establish the active state of chromatin so that multiple rounds of transcription are accomplished. While acetylation of all four core histone tails has been linked to active state of transcription, acetylation of H3 and H4 may mainly contributes to transcription of p53 response genes (5, 6, 8). In another mechanism to modulate the effect of histone acetylation, histone methylation is targeted to acetylated p53 response genes in which it transregulates the functional contribution of neighboring acetylation to p53 transcription. This is illustrated by the antagonistic action of H3-K9 methylation to H3 acetylation-mediated activation of p53 transcription. This temporal dynamic of histone acetylation status in vivo indicate that successful targeting of histone acetylation at p53 responsive promoters is accomplished by a dominant action of HAT over repressive histone-modifying activities.

Because uncontrolled HDAC/HMT activities are associated with abnormal gene repression in various diseases (9-11), the regulation of these modification processes are useful in developing drugs. Synthetic or natural inhibitors targeting specific HDAC/HMT have been explored to be effective in alleviating aberrant transcriptional repression (12-15). In most cases, these inhibitors are designed to have in vivo capability to inactivate enzymatic activities of HDACs/HMTs by binding to their catalytic sites. Although these inhibitors have been used to activate or repress distinct epigenetic pathways, their efficacy as in vivo reagents has been severely compromised by their toxic effect and short half-life. These observations underscore the need for the development of better antagonists for epigenetic factors that contribute to the metastatic potential of cancer cells. Taking into consideration antagonizing potencies of repressive HDACs/HMTs in histone acetylation signaling processes, acetylated histone tails must be physically recognized and bound by these HDAC and HMT for their negative action. This issue is of particular importance, as recent studies suggested that the transcriptional activity of p53 in chromatin is regulated by highly dynamic nature of HAT, HDAC and HMT (16, 17). Thus, utilization of inhibitors blocking substrate access of HDAC/HMT would also offer an attractive means of manipulating the acetylated state of cellular histones.

### SUMMARY

p53 is a tumor suppressor protein and activates multiple target genes upon DNA damage to promote apoptosis. p53-mediated gene activation is antagonized by histone deactylase 1 (HDAC1) and histone methyl transferase G9a. Applicant has developed acetylated H4 tail peptides which bind to HDAC 1 and G9a and impair their repressive actions on p53 target genes.

The import of peptides into human cells may be useful to selectively interfere with intracellular protein-protein interactions and thereby manipulate physiological processes (18-22). Because only a narrow range of peptides can penetrate the cell membrane by passive diffusion, interfering peptides may be conjugated to nuclear penetrating peptide (NPP) which can translocate across the membrane. NPP derived from HIV Tat is an useful tool for intracellular transport of a wide variety of different peptides (19, 21-24). There is a need for H4 tail peptides that can penetrate the cell membrance and affect the recruitment of specific chromatin-regulating factors that modulate transcriptional competence of chromatin.

Provided herein are the synthesis, cellular uptake and anti-repressive properties of H4 tail peptides. H4 tail fusion peptides can contain unmodified or acetylated H4N-terminal tail domain and a nuclear penetrating sequence such as a HIV1 TAT NPP (pTAT). The nuclear localization signal within the H4 tail domain allowed subcellular traffic of the tail peptide from the cytoplasm to the nucleus after the pTAT-mediated import into intact cells. The present functional analyses revealed that cellular import and nuclear localization of the H4 tail peptides, especially acetyl H4 peptides, yielded a significant enhancement of p53 transactivation and apoptotic cell death. More surprisingly, this increase in transcription rate was due to the sequestration of HDAC 1 and G9a activities and concomitant changes in H3/H4 acetylation and H3-K9 methylation.

Thus, in one aspect, provided herein is a method for imparing the repressive function of HDAC1 and/or G9, which in turn can increase, enhance or repair the activity of p53 in a eukaryotic cell. This activity, in turn, enhances apoptic cell death. In one aspect, the p53 dysfunction or impaired p53 function is caused by the action of histone deacetylase 1 (HDAC1) and/or histone methyl transferase (HMT or G9a) on p53. The method comprises, or alternatively consists essentially of, or yet further consists of, contacting the cell with an isolated or recombinant polypeptide comprising SEQ ID NO. 1 or 2, a biological equivalent thereof. In one aspect, the isolated or recombinant polypeptide is not lysine-acetylated in a mammalian cell system. In another aspect the polypeptide is acetylated at one or more lysine residues or replaced with an arginine (R). The polypeptide can be acetylated or substituted at one or more lysine residues by natural or chemical methods, e.g., by a commercial method available from Genemed Synthesis, Inc. In another aspect, the isolated or recombinant polypeptide further comprises, or alternatively consists essentially of, or yet further consists of, an agent that facilitates entry of the peptide into the cell (“nuclear penetrating agent”). A non-limiting example of such is a polypeptide comprising, or alternatively consisting essentially of, or yet further consisting of, SEQ ID NO. 2 or 3 or a biological equivalent thereof.

In a further aspect of this invention, SEQ ID NO. 1 or 2, a biological equivalent thereof is N-terminal to the nuclear penetrating agent. In an alternate aspect of this invention, SEQ ID NO. 1 or 2, a biological equivalent thereof is C-terminal to the nuclear penetrating agent.

In a further aspect, the methods further comprises, or alternatively consists essentially of, or yet further consists of, contacting the cell with an agent that inhibits the growth of the cell such as an anticancer drug or biologic or an agent that promotes apoptosis. Such agents are known to those of skill in the art.

In another embodiment, provided is a method of treating a condition mediated or caused by p53 dysfunction and/or impaired p53 function, comprising, alternatively consisting essentially of, or yet further consisting of, administering to the subject an effective amount of an isolated or recombinant polypeptide comprising SEQ ID NO. 1 or 2, or a biological equivalent thereof. In one aspect, the isolated or recombinant polypeptide is not lysine-acetylated in a mammalian cell system. In another aspect the polypeptide is acetylated at one or more lysine residues and/or substituted with one or more arginine (R) residues. The polypeptide can be acetylated and/or substituted at one or more lysine residues by natural or chemical methods, e.g., by a commercial method available from Genemed Synthesis, Inc.

In a yet further embodiment, provided is a method for inhibiting the growth of a cell in a subject in need thereof, comprising, or alternatively consisting of, or yet further consisting of, administering to the subject an effective amount of an isolated or recombinant polypeptide comprising SEQ ID NO. 1 or 2, a biological equivalent thereof. In one aspect, the isolated or recombinant polypeptide is not lysine-acetylated in a mammalian cell system. In another aspect the polypeptide is acetylated at one or more lysine residues and/or substituted at one or more arginine (R) residues. The polypeptide can be acetylated at one or more lysine residues and/or substituted at one or more arginine (R) residues by natural or chemical methods, e.g., by a commercial method available from Genemed Synthesis, Inc. and/or substituted at one or more arginine (R) residues

In a yet further aspect, the isolated or recombinant polypeptide further comprises, or alternatively consists essentially of, or yet further consists of, a nuclear penetrating agent that facilitates entry of the peptide into the cell in the subject. A non-limiting example of such is a polypeptide comprising, or alternatively consisting essentially of, or yet further consisting of, SEQ ID NO. 3 a biological equivalent thereof.

In a further aspect of this invention, SEQ ID NO. 1, or 2 or a biological equivalent or each thereof is N-terminal to the nuclear penetrating agent. In an alternate aspect of this invention, SEQ ID NO. 1 or 2, or a biological equivalent of each thereof is C-terminal to the nuclear penetrating agent.

In a further aspect, the method further comprises, or alternatively consists essentially of, or yet further consists of, administering to the subject an agent that inhibits the growth of the cell (i.e., a cancer cell) such as an anticancer drug or biologic, or an agent that promotes apoptosis. Such agents are known to those of skill in the art.

In each of the above embodiments, the polypeptides can be delivered by gene therapy methods which comprise, or alternatively consist essentially of, or yet further consist of, administering or contacting the cell with an isolated or recombinant polynucleotide encoding the isolated polypeptide.

Compositions for use in the methods are further provided herein. For example, an isolated or recombinant polypeptide comprising, or alternatively consisting essentially of, or yet further consisting of, SEQ ID NO. 1 or 2, or a biological equivalent thereof is provided. In one aspect the polypeptide is acetylated at one or more lysine residues and/or substituted at one or more arginine (R) residues. The polypeptide can be acetylated at one or more lysine residues and/or substituted at one or more arginine (R) residues by natural or chemical methods, e.g., by a commercial method available from Genemed Synthesis, Inc.

The isolated or recombinant polypeptide of can further comprise, or alternatively consist essentially of, or yet further consist of, a nuclear penetrating agent that facilitates entry of the polypeptide into a eukaryotic cell. Non-limiting examples of such comprises SEQ ID NO. 2 or 3, or a biological equivalent of each thereof.

In a further aspect of this invention, SEQ ID NO. 1 or 2, or a biological equivalent of each thereof is N-terminal to the nuclear penetrating agent. In an alternate aspect of this invention, SEQ ID NO. 1 or 2, or a biological equivalent of each thereof is C-terminal to the nuclear penetrating agent.

Isolated or recombinant polynucleotide encoding these polypeptides are further provided as well as isolated or recombinant polynucleotides that bind to and inhibit the expression of these polynucleotides such as agents for effecting RNA interference (RNAi) such as dsRNA, miRNA, siRNA, shRNA and antisense RNA. The isolated or recombinant polynucleotides can further comprise, or alternatively consist essentially of, or yet further consist of regulatory polynucleotide sequences operatively linked to the isolated or recombinant polynucleotide. The isolated or recombinant polynucleotides can be inserted into an expression or delivery vehicle or an isolated host cell. The host cells can be used to recombinantly produce the polypeptides by growing the host cell containing an isolated polynucleotide under conditions that favor the expression of the isolated or recombinant polynucleotide. In one aspect, the polypeptide produced from the polynucleotide is isolated from the host cell. Polynucleotides also can be replicated and isolated using this method.

The polypeptides or polynucleotides can further comprise a detectable label.

Compositions comprising a carrier and one or more of the polypeptides, polynucleotides and expression or delivery vectors are further provided herein. The carrier can be a solid support or a liquid carrier such as a pharmaceutically acceptable carrier.

Antibodies that recognize and bind an isolated or recombinant polypeptide and/or an isolated or recombinant polynucleotide are further provided. The antibodies can be used to isolate polynucleotides or polypeptides. The antibody is any of a polyclonal antibody, a monoclonal antibody, an antibody fragment such as a CDR, a chimeric antibody, an antibody derivative, a recombinant humanized antibody, recombinant antibody, a human antibody, a veneered antibody or a humanized antibody. Polynucleotides encoding the antibody and host cells containing the isolated recombinant polynucleotides are further provided by this invention. The host cells can be used to recombinantly produce the antibody polypeptides by growing the host cell containing an isolated or recombinant polynucleotide under conditions that favor the expression of the antibody expressing polynucleotide. In one aspect, the polypeptide is isolated from the host cell. Yet further provided is an antibody complex comprising the antibody of this invention and a polypeptide or polynucleotide specifically bound to the antibody. A hybridoma cell line that produces the monoclonal antibody is also provided.

Compositions comprising a carrier and one or more of the antibody or polynucleotide encoding the antibody are further provided herein. The carrier can be a solid support or a liquid carrier such as a pharmaceutically acceptable carrier.

The antibodies, fragments, and isolated polynucleotides encoding them can further comprise a detectable label.

Further provided by this invention is a kit comprising one or more of an isolated or recombinant polypeptide of this invention, an isolated or recombinant polynucleotide of this invention, or an isolated antibody or fragment thereof, of any of this invention and instructions for use. The instructions can be for use of the compositions of this invention for therapeutic, diagnostic or to screen for new therapeutic agents. In a further aspect, the kit can further comprise an agent that inhibits the growth of a cancer cell.

Screens to identify new therapeutic agents are also provided. The method comprises screening for an agent that inhibits or interferes with the action of histone deacetylase 1 (HDAC1) and/or histone methyl transferase (HMT or G9a) on p53 function in a eukaryotic cell, by contacting a eukaryotic cell expressing p53 and histone deacetylase 1 (HDAC1) and/or histone methyl transferase (HMT or G9a) with the agent and assaying for p53 function. In one aspect the cell is a cancer cell. In another aspect, the cell is a U205 cell. The screen can further comprise, or alternatively consist essentially of, or yet further consist of comparing p53 function of the cell with the ability of an isolated or recombinant polypeptide or the isolated or recombinant polynucleotide of this invention to inhibit or interfere with the action of histone deacetylase 1 (HDAC1) and/or histone methyl transferase (HMT or G9a) on p53.

Histone acetylation plays a central role in establishing an active chromatin environment. The functional contribution of histone acetylation to chromatin transcription is accomplished by a dominant action of histone acetyltransferases (HATs) over repressive histone-modifying activities at gene promoters; misregulation of these dynamic events can lead to various diseases. Here, we describe the synthesis and characterization of transducible peptides derived from histone H4N-terminal tail as a molecular tool to establish and maintain the active state of p53 target genes. Cellular experiments demonstrate a distinct increase in p53 transactivation by acetylated H4 tail peptides, but only a modest change by unmodified H4 tail peptides. The molecular basis underlying the observed effects involves the selective interaction of the tail peptides with G9a histone methyltransferase (HMT) and histone deacetylase 1 (HDAC1) and the disruption of their occupancy at p53 target promoters. Furthermore, treatment of xenograft models and cancer cell lines with the tail peptides sharply decline tumor cell growth and enhances apoptosis in response to DNA damage. These results indicate that H4 tail peptide mimics upregulate p53 transcription pathway and may be used as a novel strategy for anticancer therapy.

### BRIEF DESCRIPTION OF THE DRAWINGS

FIGS. 1A-D show H4 tail-dependent binding of HDAC 1 and G9a to nucleosomes. (A) Schematic diagram of wild type (F/H-H4), mutant (F/H-mH4) and tailless (F/H-Δ1-28H4) human H4. Four acetylatable lysine residues (underlined, light grey) are mutated to arginine (underlined, light grey). Sequences disclosed as 17, 19, 18, 19 and 19, respectively, in order of appearance. (B) Isolation of ectopic H4 mononuclesomes. Each of the histone proteins within the nucleosome was separated on 15% SDS-PAGE and visualized by Coomassie brilliant blue (CBB) staining Lane 1, wild type H4 mononucleosomes; lane 2, lysine-mutated H4 mononucleosomes; lane 3, tailless H4 mononucleosomes. The positions of endogenous histones and ectopic H4 are shown on the right. (C) Acetylation status of nucleosomal H4. Ectopic H4 nucleosomes were analyzed by Western blotting using antibodies that recognize H4 acetylated at K5, K8, K12 and/or K16. Lane 1, wild type H4 mononucleosomes; lane 2, lysine-mutated H4 mononucleosomes; lane 3, tailless H4 mononucleosomes. A fraction of F/H-intact H4 (marked with double asterisk) migrates slower than that of F/H-tailless H4 and endogenous H4 (marked with single asterisk). (D) Preferential interactions of G9a and HDAC1 with H4-acetylated nucleosomes. Proteins co-purified with the ectopic H4 nucleosomes were separated by 4-20% gradient SDS-PAGE, and the presence of several selected proteins was confirmed by Western blotting. Equal loading was assessed by anti-FLAG western blotting. Lane 1, 2% input; lane 2, wild type H4 mononucleosomes; lane 3, lysine-mutated H4 mononucleosomes; lane 4, tailless H4 mononucleosomes.

FIGS. 2A-C show stimulation of p53-mediated transcription by H4 tail peptides. (A) Amino acid sequences of H4 tail and control peptides. Peptides corresponding to the first 28 amino acids of human H4 and control (Ctrl) cationic peptides were conjugated with pTAT transmembrane carrier, derived from the HIV TAT protein (light grey). “ac” indicates acetylation of lysine residues of H4 tail domain. Sequences disclosed as SEQ ID NOS 20-22, respectively, in order of appearance. (B) Internalization of H4 tail and control peptides into the cell nucleus. U2OS cells (1×105) were treated with the FITC-labeled peptides (10 μM) for 12 h and imaged by confocal laser scanning fluorescence microscopy to ensure entry of the peptides into the cell nucleus. (C) Antirepressive effects of H4 tail peptides on p21 and Noxa genes. U2OS cells were transfected with increasing concentrations of control peptides, unmodified H4 tail peptides or acetylated H4 tail peptides for 12 h, and were treated with etoposide (100 μM) for another 12 h. p21 and Noxa mRNA levels were measured by qRT-PCR and normalized to that of β-actin.

FIGS. 3A-C show effects of H4 tail peptides on HDAC 1 and G9a promoter occupancy at p53 target genes. (A) H4 tail peptide-induced changes at the p21 promoter. U2OS cells were treated with H4 tail peptides (10 μM) and etoposide (100 μM) as in FIG. 2C, and ChIP assays of the p21 promoter region were performed with antibodies recognizing p53, HDAC1, G9a, H3 acetylation, H4 acetylation, and H3-K9 mono-/di-/tri-methylation. The results are shown as percentage of input, and the error bars indicate the means±S.E. Shown on the top is schematic diagram of p21 promoter region subjected to ChIP analysis. (B) H4 tail peptide-induced changes at the Noxa promoter. ChIP analyses were essentially as described in FIG. 3C, but over the Noxa gene promoter. (C) Selective binding of HDAC 1 and G9a to acetylated H4 tail peptides. pTAT-conjugated H4 tail peptides were examined for binding to FLAG-G9a and GST-HDAC1 immobilized on M2 agarose and glutathione sepharose beads. HDAC1/G9a-bound H4 tail peptides were detected by immunoblotting with anti-TAT antibody. Lanes 1, 3, 5, 7, 9 and 11, input peptides (5%); lanes 2 and 8, control peptide-bound factors; lanes 4 and 10, H4 tail peptide-bound factors; lanes 6 and 12, acetylated H4 tail peptide-bound factors.

FIGS. 4A-E show requirements of G9a and HDAC 1 for tail peptides-induced activation. (A) RNAi-mediated depletion of G9a and HDAC1. U2OS cells were stably transfected with control shRNA (lane 1), G9a shRNA (lane 2) or HDAC1 shRNA (lane 3), and efficiency and specificity of knockdown were confirmed by Western blotting with anti-G9a and anti-HDAC1 antibodies. β-actin was probed as a loading control. (B) G9a and HDAC1-dependent action of H4 tail peptides at p21 gene. U2OS cells stably expressing control, G9a, or HDAC1 shRNA were transfected with H4 tail peptides for 12 h and treated with etoposide (100 μM) for another 12 h. The endogenous p21 gene transcription was analyzed by qRT-PCR. The data were normalized to β-actin transcription levels. (C) G9a and HDAC1-dependent action of H4 tail peptides at Noxa gene. U2OS cells depleted of G9a or HDAC 1 were treated with etoposide and H4 tail peptides as in FIG. 4b, and Noxa gene transcription was analyzed by qRT-PCR. (D) Interdependent localization of HDAC 1 and G9a at the p21 promoter. G9a-depleted or HDAC 1-depleted cells were treated with etoposide for 12 h, and ChIP analysis was carried out essentially as described in FIG. 3A. (E) Interdependent localization of HDAC1 and G9a at the Noxa promoter. ChIP analysis was as described in FIG. 4D, but over the Noxa gene promoter.

FIGS. 5A-D show effects of H4 tail peptides on apoptosis and tumor growth. (A) Up-regulation of DNA damage-induced apoptosis by acetylated H4 tail peptides. U2OS cells (2×105) were transfected with H4 tail peptides and then treated with etoposide as described in FIG. 2C. After vigorous washing, cells were double stained with FITC-conjugated Annexin V (apoptotic fraction: lower and upper right quadrants) and PI (necrotic fraction: upper left quadrant), and apoptotic population was monitored by FACS analysis. (B) Inhibition of cell viability by acetylated H4 tail peptides. U2OS cells were transfected with H4 tail peptides and treated with etoposide as in FIG. 5a, and cell viability was measured by trypan blue staining (C) Suppression of tumor growth by H4 tail peptides. BALB/c female mice were implanted with CT26 (1×107) colon cancer cells in the right hind leg. When tumor sizes reached ˜5-6 mm in diameter on day 5, the mice were treated with local injections of H4 tail/control peptides and etoposide into xenografts. The results represent the mean tumor volume±SEM (n=7 mice per group) from two independent experiments. (D) Photographs of xenografts. Mice were euthanized 5 days after the peptide treatments, and tumors were excised.

FIG. 6 shows a model for H4 tail peptide-induced enhancement of p53 transactivation. In normal cells, G9a and HDAC1 repress p53 target genes by maintaining H3K9 methylation and histone deacetylation. Upon DNA damage, p53 target genes are activated by the competitive action of HAT against G9a and HDAC 1. The nuclear delivery of acetylated H4 tail peptides interferes with G9a and HDAC 1 activities and leads to elevated histone acetylation and increased transcriptional activity toward p53 target genes. The histone tail peptide mimics targeting chromatin remodeling factors represent a new class of chromatin-modulating agents, and could be used as experimental and pharmaceutical tools.

FIG. 7 depicts preparation of mononucleosome. 293T cells were transfected with expression vectors for wild type (lane 2), lysine mutated (lane 3) or tailless H4 (lane 4) for 48 h, and mononucleosomes were prepared by MNase digestion of chromatin. DNA fragments recovered from mononucleosomes were resolved on a 1.5% agarose gel and stained with ethidium bromide. Lane 1, 0.1-12 kb DNA ladder.

FIG. 8 depicts far-UV CD spectra of H4 tail and control peptides. CD values were determined using 50 μM unmodified/acetylated H4 tail or control peptides in the range of 190-240 nm. CD spectra are the average of 20 measurements. A largely negative ellipticity near 200 nm and no shoulder in the range 210-230 nm are typical of disordered peptides.

FIG. 9 depicts effects of H4 tail peptides on p53 modifications. U2OS cells were transfected with control peptides, unmodified tail peptides or acetylated tail peptides, and treated with etoposide as described in FIG. 2C. Cell lysates were analyzed by Western blotting using antibodies indicated on the left.

FIG. 10 depicts similar effects of H4 tail peptides in mouse CT26 cells. qRT-PCR and MTT assays were essentially as described in FIG. 2C and FIG. 5B, but using mouse CT26 colon carcinoma cells.

FIG. 11 depicts weight of tumors after 5 days of H4 tail or control peptide treatments. Mice were treated with H4 tail/control peptides and etoposide as in FIG. 5C. The weights of the dissected tumors were measured and expressed as mean±SEM.

### DETAILED DESCRIPTION

The p53 tumor suppressor is a key component of cellular mechanisms that maintain genomic integrity after DNA damage (1, 2). p53 regulates these processes mainly, if not solely, by functioning as a sequence-specific transcription factor that stimulates expression of a number of target genes (3, 4). Recent studies revealed that p53 transcriptional activity in the context of chromatin requires the recruitment of chromatin remodeling machineries to target genes (5-8, 29). Among diverse chromatin remodeling processes, acetylation of histone N-terminal tails has been implicated in p53-mediated transcription (5, 6, 8, 29). When cells are exposed to stress conditions, p53 recruits HAT and establishes distinct histone acetylation at its target genes. Enhanced acetylation levels in turn permit the transcriptional machinery to access the promoters and initiate the high level of transcription (5, 6, 8, 29). Histone acetylation is a reversible process, which is dynamically governed by the opposing activities of HAT and HDAC. For the initiation of p53-mediated transactivation, HAT activity dominates over HDAC activity to establish promoter-targeted histone acetylation that ultimately results in active transcription. Although acetylation of all four core histones has been linked to transcriptional activation, recent studies indicate that acetylation of H3 and H4 plays a more important role in p53 target gene expression (5, 6, 8, 29). Besides histone acetylation, histone methylation has also been linked to regulation of p53-induced transcriptional events. This is well-illustrated by the repressive effects of H3K9 methylation on p53-mediated transactivation (5, 16). Along with indications that the effects of H3/H4 acetylation on transcriptional activity are disrupted by H3K9 methylation, established histone acetylation state appears to act on transcription only for short time frame. Such dynamics of histone acetylation allows the assumption that the steady state level of histone acetylation at p53 responsive promoters is accomplished by a dominant action of HAT over repressive histone-modifying activities.

Since misregulation of histone acetylation/methylation processes leads to aberrant gene expression in various diseases (9-11), synthetic or natural inhibitors targeting specific HDAC/HMT have been explored (12-15). In most cases, inhibitors are designed to bind to the catalytic sites of HDACs/HMTs and inactivate their enzymatic activities. Although these inhibitors would affect distinct epigenetic reactions, their efficacy as cellular reagents is severely compromised by their toxicity and short half-life. These drawbacks underscore the need for the development of more effective strategies to control HDAC/HMT activities. Taking into consideration that HDACs and some HMTs mainly target acetylated nucleosomes for their repressive actions, they should be able to physically recognize histone acetylation. This issue is of particular importance for p53 function, as recent studies demonstrated that p53 transactivation is regulated by dynamic balance between histone acetylation and histone deacetylation/methylation (16, 17). Thus, utilization of inhibitors blocking substrate access of HDAC/HMT would offer an attractive means of manipulating p53 transcription pathway.

Related to the current report, the import of specific peptides into human cells has been employed to interfere with intracellular protein-protein interactions and thereby manipulate distinct physiological processes (18-22). Since only a narrow range of peptides can penetrate the cell membrane by passive diffusion, interfering peptides are often conjugated to cell-penetrating peptide (CPP). Despite our lack of understanding of what the endogenous processes are, CPP derived from HIV TAT is proven to be extremely useful for intracellular transport of synthetic peptides (19, 21-24). Given the increasing utility of cell-penetrating peptides for biomedical applications, we became interested in exploring whether histone tail peptides have any effects on the recruitment of specific factors that modulate transcriptional competence of chromatin.

Here, Applicant shows that H4 tail fusion peptides containing H4N-terminal tail domain and an HIV TAT CPP (pTAT) stimulate p53-mediated transactivation. The nuclear localization signal within the H4 tail domain allows subcellular traffic of the tail peptides from the cytoplasm to the nucleus after the pTAT-mediated import into living cells. The fact that acetylated H4 tail peptides activate transcription more efficiently than do the unmodified peptides supports the specificity of the observed effects. Importantly, cellular import and nuclear localization of H4 tail peptides sequestrate G9a and HDAC1 activities, adding mechanistic insight into the observed enhancement of p53 transactivation.

### DEFINITIONS

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated, that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “subject” of diagnosis or treatment is a cell or an animal such as a mammal, or a human. Non-human animals subject to diagnosis or treatment are, for example, simians, murine, such as, rats, mice, canine, such as dogs, leporids, such as rabbits, livestock, such as bovine or ovine, sport animals, such as equine, and pets, such as canine and feline.

A “chimeric polypeptide”, “chimeric protein” or “fusion protein” refers to a protein, peptide or polypeptide created through the joining of two or more amino acid sequences or alternatively created by expression of a joint nucleotide sequence comprising two or more nucleotide sequences which originally code for separate proteins, peptides, polypeptides. Translation of joined nucleotide sequence, also known as a fusion gene, results in a single polypeptide, the “chimeric polypeptide”, with functional properties derived from each of the original proteins.

A “nuclear penetrating agent” such as a “nuclear penetrating peptide” or “NPP” intends a short peptide that facilitates cellular uptake of a molecular cargo. A molecular cargo can be any chemical or biological molecule or complex ranging from a small chemical molecule to nanosize particles and large fragments of DNA or polypeptides. The molecular cargo is associated with the peptides either through chemical linkage via covalent bonds or through non-covalent interactions. NPPs typically have an amino acid composition containing either a high relative abundance of positively charged amino acids such as lysine or arginine, or have sequences that contain an alternating pattern of polar/charged amino acids and non-polar, hydrophobic amino acids. A non-limiting example of a nuclear penetrating peptide is the NPP of the trans-activating transcriptional activator (Tat) from Human Immunodeficiency Virus 1 (HIV-1) (reviewed in Wagstaff et al. (2006) Current Medicinal Chemistry 13(12) 1371-87). Since the discovery of Tat, the number of known NPPs has expanded considerably and small molecule synthetic analogues with more effective protein transduction properties have been generated, such as those disclosed in Okuyama et al. (2007) Nature Methods 4(2)153-159, U.S. Patent Publication Nos. US2008/0234183, US2010/0048487, US2010/0061932, US2009/0036363 and U.S. Pat. No. 7,579,318. The term nuclear penetrating agent intends peptides and small molecule synthetic analogues.

The terms “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, interfering RNA (RNAi), messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Short interfering RNA” (siRNA) refers to double-stranded RNA molecules (dsRNA), generally, from about 10 to about 30 nucleotides in length that are capable of mediating RNA interference (RNAi), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA.

“Double stranded RNA” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

A siRNA can be designed following procedures known in the art. See, e.g., Dykxhoorn, D. M. and Lieberman, J. (2006) “Running Interference: Prospects and Obstacles to Using Small Interfering RNAs as Small Molecule Drugs,” Annu Rev. Biomed. Eng. 8:377-402; Dykxhoorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering with disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; U.S. Patent Application Publication No. 2008/0188430; and U.S. Patent Application Publication No. 2008/0249055.

siRNAs can be made with methods known in the art. See, e.g., Dykxhoorn, D. M. and Lieberman, J. (2006) “Running Interference: Prospects and Obstacles to Using Small Interfering RNAs as Small Molecule Drugs,” Annu Rev. Biomed. Eng. 8:377-402; Dykxhoorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering with disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; U.S. Patent Application Publication No. 2008/0188430; and U.S. Patent Application Publication No. 2008/0249055.

A siRNA may be chemically modified to increase its stability and safety. See, e.g. Dykxhoorn, D. M. and Lieberman, J. (2006) “Running Interference: Prospects and Obstacles to Using Small Interfering RNAs as Small Molecule Drugs,” Annu Rev. Biomed. Eng. 8:377-402 and U.S. Patent Application Publication No. 2008/0249055.

“shRNA” is short hairpin RNA.

microRNA or miRNA are single-stranded RNA molecules of 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

A siRNA vector, dsRNA vector or miRNA vector as used herein, refers to a plasmid or viral vector comprising a promoter regulating expression of the RNA. “siRNA promoters” or promoters that regulate expression of siRNA, dsRNA, or miRNA are known in the art, e.g., a U6 promoter as described in Miyagishi and Taira (2002) Nature Biotech. 20:497-500, and a H1 promoter as described in Brummelkamp et al. (2002) Science 296:550-3.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include recombinant polynucleotides and nucleic acid fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together. An example of such is the hybrid polypeptide of SEQ ID NO. 2 and the polynucleotide that encodes it.

As used herein, the term “biological equivalent thereof” is used synonymously with “equivalent” unless otherwise specifically intended. When referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 60%, or 65%, or 70%, or 75%, or 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Alternatively, a biological equivalent is a peptide encoded by a nucleic acid that hybridizes under stringent conditions to a nucleic acid or complement that encodes the peptide. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide or polynucleotide region (or a polypeptide or polypeptide region) having a certain percentage (for example, about 60%, 65%, 70%, 75%, 80%, 85%, 90%, 95% or 97%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

An “equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Regulatory polynucleotide sequences” intends any one or more of promoters, operons, enhancers, as know to those skilled in the art to facilitate and enhance expression of polynucleotides.

An “expression vehicle” is a vehicle or a vector, non-limiting examples of which include viral vectors or plasmids, that assist with or facilitate expression of a gene or polynucleotide that has been inserted into the vehicle or vector.

A “delivery vehicle” is a vehicle or a vector that assists with the delivery of an exogenous polynucleotide into a target cell. The delivery vehicle may assist with expression or it may not, such as traditional calcium phosphate transfection compositions.

A “composition” is intended to mean a combination of active agent and another compound or composition, inert (for example, a solids support or pharmaceutically acceptable carrier) or active, such as an adjuvant.

A “pharmaceutical composition” is intended to include the combination of an active agent with a carrier, inert or active, making the composition suitable for diagnostic or therapeutic use in vitro, in vivo or ex vivo.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

As used herein, to “treat” further includes systemic amelioration of the symptoms associated with the pathology and/or a delay in onset of symptoms. Clinical and sub-clinical evidence of “treatment” will vary with the pathology, the subject and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, topical application, intrapentoneal, intravenous and by inhalation. An agent of the present invention can be administered for therapy by any suitable route of administration. It will also be appreciated that the preferred route will vary with the condition and age of the recipient, and the disease being treated.

The agents and compositions of the present invention can be used in the manufacture of medicaments and for the treatment of humans and other animals by administration in accordance with conventional procedures, such as an active ingredient in pharmaceutical compositions.

The term “conjugated moiety” refers to a moiety that can be added to an isolated polypeptide by forming a covalent bond with a residue of polypeptide. The moiety may bond directly to a residue of the polypeptide or may form a covalent bond with a linker which in turn forms a covalent bond with a residue of the polypeptide.

A “peptide conjugate” refers to the association by covalent or non-covalent bonding of one or more polypeptides and another chemical or biological compound. In a non-limiting example, the “conjugation” of a polypeptide with a chemical compound results in improved stability or efficacy of the polypeptide for its intended purpose. In one embodiment, a peptide is conjugated to a carrier, wherein the carrier is a liposome, a micelle, or a pharmaceutically acceptable polymer.

As used herein, the term “detectable label” intends a directly or indirectly detectable compound or composition that is conjugated directly or indirectly to the composition to be detected, e.g., N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Liposomes” are microscopic vesicles consisting of concentric lipid bilayers that are suitable expression or delivery vehicles. Structurally, liposomes range in size and shape from long tubes to spheres, with dimensions from a few hundred Angstroms to fractions of a millimeter. Vesicle-forming lipids are selected to achieve a specified degree of fluidity or rigidity of the final complex providing the lipid composition of the outer layer. These are neutral (cholesterol) or bipolar and include phospholipids, such as phosphatidylcholine (PC), phosphatidylethanolamine (PE), phosphatidylinositol (PI), and sphingomyelin (SM) and other types of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanol-amine, phosphatidylserine, phosphatidylinositol, sphingomyelin, cephalin, cardiolipin, phosphatidic acid, cerebrosides, distearoylphosphatidylethan-olamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carb-oxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioleoylphosphatidylglycerol and (DOPG), dicetylphosphate that are able to form vesicles. Typically, liposomes can be divided into three categories based on their overall size and the nature of the lamellar structure. The three classifications, as developed by the New York Academy Sciences Meeting, “Liposomes and Their Use in Biology and Medicine,” December 1977, are multi-lamellar vesicles (MLVs), small uni-lamellar vesicles (SUVs) and large uni-lamellar vesicles (LUVs).

A “micelle” is an aggregate of surfactant molecules dispersed in a liquid colloid. A micelle is an example of a delivery or expression vehicle. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the head groups at the center with the tails extending out (water-in-oil micelle). Micelles can be used to attach a polynucleotide, polypeptide, antibody or composition described herein to facilitate efficient delivery to the target cell or tissue.

The phrase “pharmaceutically acceptable polymer” refers to the group of compounds which can be conjugated to one or more polypeptides described here. It is contemplated that the conjugation of a polymer to the polypeptide is capable of extending the half-life of the polypeptide in vivo and in vitro. Non-limiting examples include polyethylene glycols, polyvinylpyrrolidones, polyvinylalcohols, cellulose derivatives, polyacrylates, polymethacrylates, sugars, polyols and mixtures thereof.

“Pharmaceutically acceptable carriers” refers to any diluents, excipients, or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

A “gene delivery vehicle” is defined as any molecule that can carry inserted polynucleotides into a host cell. Examples of gene delivery vehicles are liposomes, micelles pharmaceutically acceptable polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this invention can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmic vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacteria produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearised by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this invention. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

Examples of solid phase supports include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells. Non-limiting examples include simian, bovine, ovine, porcine, murine, rats, canine, equine, feline, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to prokaryotic Cyanobacteria, bacillus bacteria, E. coli bacterium, and Salmonella bacterium.

As used herein, an “antibody” includes whole antibodies and any antigen binding fragment or a single chain thereof. Thus the term “antibody” includes any protein or peptide containing molecule that comprises at least a portion of an immunoglobulin molecule, i.e., an antibody fragment. Examples of antibody fragments include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The antibodies can be polyclonal or monoclonal and can be isolated from any suitable biological source, e.g., murine, rat, sheep and canine.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

The terms “monoclonal antibody” or “monoclonal antibody composition” as used herein refer to a preparation of antibody molecules of single molecular composition from a hybridoma. A monoclonal antibody composition displays a single binding specificity and affinity for a particular epitope.

### Descriptive Embodiments

### Therapeutic Methods

The present invention provides a method for imparing the repressive function of HDAC1 and/or G9, which in turn can increase or enhance or repair the activity of p53 in a eukaryotic cell and/or enhance or upregulate p53 transcription pathway and therefore a strategy for anticancer therapy. In one aspect, the p53 dysfunction or impaired p53 function is caused by the action of histone deacetylase 1 (HDAC1) and/or histone methyl transferase (HMT or G9a) on p53. The method comprises, or alternatively consists essentially of, or yet further consists of, contacting the cell with an isolated or recombinant polypeptide comprising or alternatively consisting essentially of, or yet further consisting of, SEQ ID NO. 1 or 2, or a biological equivalent of each thereof. In one aspect, the isolated or recombinant polypeptide is not lysine-acetylated in a mammalian cell system, or alternatively by delivering an isolated or recombinant polynucleotide encoding the polypeptide. In another aspect, the isolated or recombinant polypeptide further comprises, or alternatively consists essentially of, or yet further consists of, an agent that facilitates entry of the polypeptide into the cell and/or an unnatural amino acid or label. A non-limiting example of such is an isolated or recombinant polypeptide comprising, or alternatively consisting essentially of, or yet further consisting of, SEQ ID NO. 3 or a biological equivalent thereof and/or further wherein the polypeptide comprises an unnatural amino acid or label. In a further aspect of this invention, SEQ ID NO. 1 or 2 or a biological equivalent thereof is N-terminal to the nuclear penetrating agent. In an alternate aspect of this invention, SEQ ID NO. 1 or 2 or a biological equivalent thereof is C-terminal to the nuclear penetrating agent. In one particular aspect, the cell is a cancer cell. Thus, in a further aspect, the method further comprises, or alternatively consists essentially of, or yet further consists of, contacting the cancer cell with an agent that inhibits the growth of the cancer cell such as an anticancer drug or biologic. Such agents are known to those of skill in the art, e.g., 5-fluorouracil, a platinum drug, such as cisplatinum, oxaliplatinum, a topoisomerase inhibitor, cetuximab, or Avastin.

In another embodiment, this invention provides a method of treating a condition in a subject in need thereof and mediated by p53 dysfunction and/or impaired p53 function, comprising, or alternatively consisting essentially of, or yet further consisting of, administering to the subject an effective amount of an isolated or recombinant polypeptide comprising or alternatively consisting essentially of, or yet further consisting of SEQ ID NO. 1 or 2, a biological equivalent thereof and/or further wherein the polypeptide comprises an unnatural amino acid or label. In one aspect, the isolated or recombinant polypeptide is not lysine-acetylated in a mammalian cell system. In another aspect, the isolated peptide further comprises, or alternatively consists essentially of, or yet further consists of, an agent that facilitates entry of the peptide into the cell. A non-limiting example of such is a polypeptide comprising, or alternatively consisting essentially of, or yet further consisting of, SEQ ID NO. 3 or a biological equivalent thereof and/or further wherein the polypeptide comprises an unnatural amino acid or label. In a further aspect of this invention, SEQ ID NO. 1 or 2 or a biological equivalent of each thereof is N-terminal to the nuclear penetrating agent. In an alternate aspect of this invention, SEQ ID NO. 1 or 2 or a biological equivalent of each thereof is C-terminal to the nuclear penetrating agent. The method can also be practiced by delivering to the cell a polynucleotide encoding the polypeptide. In one particular aspect, the cell is a cancer cell. Thus, in a further aspect, the methods further comprises, or alternatively consists essentially of, or yet further consists of, contacting the cancer cell with an agent that inhibits the growth of the cancer cell such as an anticancer drug or biologic. Such agents are known to those of skill in the art.

In a yet further embodiment, this invention provides a method of inhibiting the growth of a eukaryotic cell in a subject in need thereof, comprising, or alternatively consisting of, or yet further consisting of, administering to the subject an effective amount of an isolated or recombinant polypeptide comprising or alternatively consisting essentially of, or yet further consisting of, SEQ ID NO. 1 or 2 or a biological equivalent of each thereof and/or further wherein the polypeptide comprises an unnatural amino acid or label. In one aspect, the isolated or recombinant polypeptide is not lysine-acetylated in a mammalian cell system. In another aspect, the isolated peptide further comprises, or alternatively consists essentially of, or yet further consists of, an agent that facilitates entry of the polypeptide into the cell. A non-limiting example of such is a polypeptide comprising, or alternatively consisting essentially of, or yet further consisting of, SEQ ID NO. 3 or a biological equivalent thereof and/or further wherein the polypeptide comprises an unnatural amino acid or label. The method can also be practiced by delivering to the cell a polynucleotide encoding the polypeptide. In a further aspect of this invention, SEQ ID NO. 1 or 2, or a biological equivalent thereof is N-terminal to the nuclear penetrating agent. In an alternate aspect of this invention, SEQ ID NO. 1 or 2 a biological equivalent thereof is C-terminal to the nuclear penetrating agent. In one particular aspect, the cell is a cancer cell. Thus, in a further aspect, the method further comprises, or alternatively consists essentially of, or yet further consists of, contacting the cancer cell with an agent that inhibits the growth of the cancer cell such as an anticancer drug or biologic. Such agents are known to those of skill in the art, e.g., 5-fluorouracil, a platinum drug, a topoisomerase inhibitor, cetuximab, or Avastin.

For the above noted methods, a non-limited example of a biological equivalent of SEQ ID NO. 1 or 2, includes a fragment of SEQ ID NO. 1 or 2, wherein the polypeptide is independently deleted at the amine and/or carboxyl terminus by at least 1, or alternatively at least 2, or alternatively at least 3, or alternatively at least 4, or alternatively at least 5, or alternatively at least 6, or alternatively at least 7, or alternatively at least 8, or alternatively at least 9, or alternatively at least 10 amino acids of the wild-type sequence. Alternatively, additional amino acids can be added to the amine and/or carboxyl terminus which may or may not correspond to the wild type sequence, of by at least 1, or alternatively at least 2, or alternatively at least 3, or alternatively at least 4, or alternatively at least 5, or alternatively at least 6, or alternatively at least 7, or alternatively at least 8, or alternatively at least 9, or alternatively at least 10 amino acids.

For the above noted methods, a non-limited example of a nuclear penetrating sequence comprises, or alternatively consisting essentially of, or yet further consisting of, SEQ ID NO. 3 or an equivalent thereof. Equivalents thereof include a fragment of SEQ ID NO. 3, wherein the polypeptide is independently deleted at the amine and/or carboxyl terminus by at least 1, or alternatively at least 2, or alternatively at least 3, or alternatively at least 4, or alternatively at least 5, or alternatively at least 6, or alternatively at least 7, or alternatively at least 8, or alternatively at least 9, or alternatively at least 10 amino acids of the wild-type sequence. Alternatively, additional amino acids can be added to the amine and/or carboxyl terminus which may or may not correspond to the wild type sequence, of by at least 1, or alternatively at least 2, or alternatively at least 3, or alternatively at least 4, or alternatively at least 5, or alternatively at least 6, or alternatively at least 7, or alternatively at least 8, or alternatively at least 9, or alternatively at least 10 amino acids.

In a further aspect of this invention, SEQ ID NO. 1 or 2 or a biological equivalent thereof is N-terminal to the nuclear penetrating agent. In an alternate aspect of this invention, SEQ ID NO. 1 or a biological equivalent thereof is C-terminal to the nuclear penetrating agent. In addition, for the above-noted methods, the recombinant or isolated polypeptide of SEQ ID NO. 1 or 2 may lack acetylation at all four lysines, or alternatively be lysine-acetylated or arginine substituted at 1, 2, 3 or all 4 lysine residues. Further provided for use in the methods of this invention is an isolated or recombinant polypeptide consisting essentially of the amino acid sequence SGRGXGGXGL GXGGAXRHRK VLRDNIQG (SEQ ID NO: 23) wherein X is independently the same or different and is lysine or a side chain acetylated lysine and/or arginine substituted.

The methods as described herein are particularly useful in the treatment of neoplastic diseases such as cancer and certain non-neoplastic diseases. Genetic abnormality of p53 has been observed in basically all cancer types, including lung cancer, stomach cancer, breast cancer, colon cancer, liver cancer, prostate cancer, cervix/uteri cancer, head and neck cancer, esophageal cancer, leukemia, lymphoma, ovarian cancer and bladder cancer (Soussi et al. (2005) Hum Mutat 25: 6-17). The restoration of p53 function, therefore, may be helpful in the treatment of these cancers and therefore this invention provides therapies for their treatment. Non-neoplastic diseases medicated by dysfunctional p53 include, for example, atherosclerosis and neurological diseases such as ataxias and Huntington's disease (Royds and Iacopetta (2006) Cell Death and Differentiation 13:1017-26). Enhanced p53 achieved by the methods of the current invention, therefore, is also helpful with the treatment of these non-neoplastic diseases.

### Polypeptides

Applicant also provides an isolated or recombinant polypeptide comprising, or alternatively consisting essentially of, or yet further consisting of, SEQ ID NO. 1 or 2 or a biological equivalent thereof and/or further wherein the polypeptide comprises an unnatural amino acid or label. In one aspect, the polypeptide is not lysine-acetylated in a mammalian cell system. In a further aspect, the isolated or recombinant polypeptide is acetylated at one or more, but not all available lysines, e.g., two or more, three or all four lysines shown in SEQ ID NO. 1 or 2 and/or further wherein the polypeptide comprises an unnatural amino acid or label. Further provided is an isolated or recombinant polypeptide consisting essentially of the amino acid sequence SGRGXGGXGL GXGGAXRHRK VLRDNIQG (SEQ ID NO: 23) wherein X is independently the same or different and is lysine or a side chain acetylated lysine and/or an arginine. The polypeptides can be recombinantly or chemically synthesized and can further comprise post-translational modification.

In another aspect, the isolated or recombinant polypeptide further comprises, or alternatively consists essentially of, or yet further consists of, an isolated or recombinant polypeptide that facilitates entry of the peptide into the cell. A non limiting example of such is an amino acid sequence that comprises, or alternatively consists essentially of, or yet further consists of SEQ ID NO. 3 or a biological equivalent thereof.

The isolated polypeptide as described herein may further comprise, or alternatively consist essentially of, or yet further consist of, at least one of a protein start site and/or a polyhistidine tag each operatively linked to the polypeptide.

The polypeptides can further comprise a detectable label. Such labels are known to those of skill in the art and examples of such are described herein.

An isolated host cell that comprises, or alternatively consists essentially of, or yet further consists of the isolated or recombinant polypeptide as described herein, is further provided. The isolated host cells can be a prokaryotic or a eukaryotic cell. Suitable cells containing the inventive polypeptides include prokaryotic and eukaryotic cells, which include, but are not limited to bacterial cells, algae cells, yeast cells, insect cells, plant cells, animal cells, mammalian cells, murine cells, rat cells, sheep cells, simian cells and human cells. A non-limiting example of algae cells is red alga Griffithsia sp. from (Toshiyuki et al. (2005) J. Biol. Chem. 280(10):9345-53). A non-limiting example of plant cells is a Nicotiana benthamiana leaf cell (O'Keefe (2009) Proc. Nat. Acad. Sci. USA 106(15):6099-6104). Examples of bacterial cells include Escherichia coli (Giomarelli et al. (2006), supra), Salmonella enteric, Streptococcus gordonii and lactobacillus (Liu et al. (2007) Cellular Microbiology 9:120-130; Rao et al. (2005) PNAS 102:11993-11998; Chang et al. (2003) PNAS 100(20):11672-11677; Liu et al. (2006) Antimicrob. Agents & Chemotherapy 50(10):3250-3259). The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line CHO, BHK-21; the murine cell lines designated NIH3T3, NS0, C 127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

Compositions are also provided. The compositions comprise, or alternatively consist essentially of, or yet further consist of, a carrier and the isolated or recombinant polypeptide as described herein. The carrier can be a solid support or a liquid carrier such as a pharmaceutically acceptable carrier.

Polypeptides comprising the amino acid sequences of the invention can be prepared by expressing polynucleotides encoding the polypeptide sequences of this invention in an appropriate host cell. This can be accomplished by methods of recombinant DNA technology known to those skilled in the art. Accordingly, this invention also provides methods for recombinantly producing the polypeptides of this invention in a eukaryotic or prokaryotic host cells, as well as the isolated host cells used to produce the proteins. The proteins and polypeptides of this invention also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence. The polypeptides can be chemically acetylated.

It is known to those skilled in the art that modifications can be made to any peptide to provide it with altered properties. Polypeptides of the invention can be modified to include one or more acetylated lysines and/or to unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the invention as retro-inverso peptides. In addition, the present invention envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present invention provides a method to produce a constrained peptide that incorporates random sequences at all other positions.

Non-classical amino acids may be incorporated in the peptides of the invention in order to introduce particular conformational motifs, examples of which include without limitation: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski & Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai & Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are know to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington D.C.; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach & Hasegawa (1996) J. Mol. Evol. 42:459-468; the block substitution matrices (BLOSUM) as described by Henikoff & Henikoff (1992) Proc. Natl. Acad. Sci. USA 89:1-1; Poisson models as described by Nei (1987) Molecular Evolutionary Genetics. Columbia University Press, New York.; and the Maximum Likelihood (ML) Method as described by Müller et al. (2002) Mol. Biol. Evol. 19:8-13.

### Antibody Compositions

This invention also provides an antibody capable of specifically forming a complex with a polypeptide or polynucleotide of this invention, which are useful in the screens of this invention. The term “antibody” includes polyclonal antibodies and monoclonal antibodies, antibody fragments, as well anti-idiotypic, humanized, chimeric, and recombinant antibodies (described above). The antibodies include, but are not limited to mouse, rat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify polypeptides.

This invention also provides an antibody-peptide complex comprising, or alternatively consisting essentially of, or yet alternatively consisting of, antibodies described above and a polypeptide or polynucleotide specifically bound to the antibody. In one aspect the polypeptide is the polypeptide against which the antibody was raised. In one aspect the antibody-peptide complex is an isolated complex. In a further aspect, the antibody of the complex is, but not limited to, a polyclonal antibody, a monoclonal antibody, an antibody fragment, a humanized antibody or an antibody derivative described herein. Either or both of the antibody or peptide of the antibody-peptide complex can be detectably labeled or further comprises a detectable label conjugated to it. In one aspect, the antibody-peptide complex of the invention can be used as a control or reference sample in diagnostic or screening assays.

Polyclonal antibodies of the invention can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammals serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antiben depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com., last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

In one embodiment, the antibodies described herein can be generated using a Multiple Antigenic Peptide (MAP) system. The MAP system utilizes a peptidyl core of three or seven radially branched lysine residues, on to which the antigen peptides of interest can be built using standard solid-phase chemistry. The lysine core yields the MAP bearing about 4 to 8 copies of the peptide epitope depending on the inner core that generally accounts for less than 10% of total molecular weight. The MAP system does not require a carrier protein for conjugation. The high molar ratio and dense packing of multiple copies of the antigenic epitope in a MAP has been shown to produce strong immunogenic response. This method is described in U.S. Pat. No. 5,229,490 and is herein incorporated by reference in its entirety.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1196) Proc. Natl. Acad. Sci. USA 93:7843-7848); gel microdroplet and flow cytometry (Powell et al. (1990) Biotechnol. 8:333-337; One Cell Systems, (Cambridge, Mass.).; Gray et al. (1995) J. 1 mm. Meth. 182:155-163; and Kenny et al. (1995) Bio. Technol. 13:787-790); B-cell selection (Steenbakkers et al. (1994) Molec. Biol. Reports 19:125-134.

Antibody derivatives of the present invention can also be prepared by delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” also includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody derivatives also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present invention can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 68(4):1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Delivery Reviews 31:33-42; Green & Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this invention also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this invention can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al. (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}-C_{H}1-VH-C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

If a monoclonal antibody being tested binds with protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al. (1985) Proc. Natl. Acad. Sci. USA 82:8653 or Spira et al. (1984) J. Immunol. Methods 74:307.

The isolation of other hybridomas secreting monoclonal antibodies with the specificity of the monoclonal antibodies of the invention can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

In some aspects of this invention, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow & Lane (1988) supra.

The antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

### Isolated or Recombinant Polynucleotides

Further provided is an isolated or recombinant polynucleotide encoding the isolated polypeptide or an interfering polynucleotide as described above as well as a vector or other polynucleotide construct comprising a delivery vehicle or vector and a polynucleotide of this invention. Non-limiting examples of delivery vehicles include a plasmid, a yeast artificial chromosome, a liposome, a micelle, or a viral vector.

This invention further provides isolated or recombinant polynucleotides that encode the polypeptides of this invention or for use in the methods of this invention as well as isolated or recombinant polynucleotides that bind to and inhibit the expression of these polynucleotides such as agents for effecting RNA interference (RNAi) such as dsRNA, miRNA, siRNA, shRNA and antisense RNA. Yet another aspect of the invention provides an isolated polynucleotide encoding for an antibody or a fragment of the antibody of the invention.

The isolated polynucleotides can further comprise, or alternatively consist essentially of, or yet further consist of regulatory polynucleotide sequences operatively linked to the isolated polynucleotide. The isolated polynucleotides can be inserted into an expression or delivery vehicle or an isolated host cell. The host cells can be used to recombinantly produce the polypeptides by growing the host cell containing an isolated polynucleotide under conditions that favor the expression of the isolated polynucleotide. In one aspect, the polypeptide produced from the polynucleotide is isolated from the host cell. Also provided is a DNA construct comprising an expression or delivery vehicle and a polynucleotide. In one aspect, the vector is a plasmid vector, a yeast artificial chromosome, or a viral vector. In one aspect, the vector comprises a protein tag. Protein tags can be selected from a GST-tag, a myc-tag, or a FLAG-tag provided in expression constructs commercially available from, e.g., Invitrogen, Carlsbad, Calif.

Another aspect of the invention provides an isolated host cell transformed with a polynucleotide or a DNA construct of the invention. The isolated host cells can be a prokaryotic or a eukaryotic cell. Yet another aspect of the invention provides an isolated transformed host cell expressing an isolated polypeptide, an antibody or a fragment of the antibody of the invention. The isolated host cells can be a prokaryotic or a eukaryotic cell. Suitable cells containing the inventive polypeptides include prokaryotic and eukaryotic cells, which include, but are not limited to bacterial cells, algae cells, yeast cells, insect cells, plant cells, animal cells, mammalian cells, murine cells, rat cells, sheep cells, simian cells and human cells. A non-limiting example of algae cells is red alga Griffithsia sp. from (Toshiyuki et al. (2005) J. Biol. Chem. 280(10):9345-53). A non-limiting example of plant cells is a Nicotiana benthamiana leaf cell (O'Keefe (2009) Proc. Nat. Acad. Sci. USA 106(15):6099-6104). Examples of bacterial cells include Escherichia coli (Giomarelli et al. (2006), supra), Salmonella enteric, Streptococcus gordonii and lactobacillus (Liu et al. (2007) Cellular Microbiology 9:120-130; Rao et al. (2005) PNAS 102:11993-11998; Chang et al. (2003) PNAS 100(20):11672-11677; Liu et al. (2006) Antimicrob. Agents & Chemotherapy 50(10):3250-3259). The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line CHO, BHK-21; the murine cell lines designated NIH3T3, NS0, C 127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

Also provided are polynucleotides encoding substantially homologous and biologically equivalent polypeptides to the inventive polypeptides and polypeptide complexes. Substantially homologous and biologically equivalent intends those having varying degrees of homology, as described above. It should be understood although not always explicitly stated that embodiments to substantially homologous polypeptides and polynucleotides are intended for each aspect of this invention, e.g., polypeptides, polynucleotides and antibodies.

The polynucleotides of this invention can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this invention also provides a process for obtaining the polynucleotides of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

The polynucleotides can be detectably labeled.

### Host Cells and Compositions

Also provided by this invention is one or more of an isolated or recombinant polypeptide, an antibody, or an isolated or recombinant polynucleotide or host cell further comprising a detectable label as described above. Non-limiting examples of detectable labels include a protein, an enzyme, a protein tag, or a radioisotope.

Any of the above noted polypeptide, polynucleotide, antibody or host cell can be further combined with a carrier, excipient, or diluent. In one aspect, the carrier, excipient, or diluent is pharmaceutically acceptable. The carrier can be a solid phase carrier, a gel, an aqueous liquid carrier, a paste, a liposome, a micelle, albumin, polyethylene glycol, a pharmaceutically acceptable polymer, or a pharmaceutically acceptable carrier, such a phosphate buffered saline.

The compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Compositions may optionally contain stabilizers, pH modifiers, surfactants, bioavailability modifiers and combinations of these.

Compositions may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension compositions may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension compositions.

The compositions of this invention are formulated for pharmaceutical administration to a mammal, preferably a human being. Such compositions of the invention may be administered in a variety of ways, preferably topically or by injection.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the invention. It should be understood that a specific dosage and treatment regimen for any particular subject will depend upon a variety of factors, including the activity of the specific antidote employed, the age, body weight, general health, sex and diet, renal and hepatic function of the subject, and the time of administration, rate of excretion, drug combination, judgment of the treating physician or veterinarian and severity of the particular disease being treated.

Another aspect of the invention provides a peptide conjugate comprising, or alternatively consisting essentially of, or alternatively consisting of, a carrier covalently or non-covalently linked to an isolated polypeptide of the invention. In some embodiments, the carrier comprises a liposome, or alternatively a micelle, or alternatively a pharmaceutically acceptable polymer, or a pharmaceutically acceptable carrier.

The polypeptides and polypeptide conjugates of the invention can be used in a variety of formulations, which may vary depending on the intended use. For example, one or more can be covalently or non-covalently linked (complexed) to various other molecules, the nature of which may vary depending on the particular purpose. For example, a peptide of the invention can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the invention can be complexed covalently or non-covalently with a solid support, a variety of which are known in the art and described herein. An antigenic peptide epitope of the invention can be associated with an antigen-presenting matrix such as an MHC complex with or without co-stimulatory molecules.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartrate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio) propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of common hetero-bifunctional cross-linking agents that may be used to effect the conjugation of proteins to peptides include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl)cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Cross-linking may be accomplished by coupling a carbonyl group to an amine group or to a hydrazide group by reductive amination.

The polypeptides or polynucleotides of the compositions of the invention also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

### Kits

An aspect of the invention provides a kit for use in increasing p53 activity in a eukaryotic cell comprising, or alternatively consisting essentially of, or alternatively consisting of, one or more of an isolated or recombinant polypeptide, an isolated or recombinant polynucleotide or an antibody of the invention and instructions to use.

Also provided is a kit for use in treating a subject in need thereof, comprising, or alternatively consisting essentially of, or alternatively consisting of, an isolated or recombinant polypeptide or polynucleotide or a composition of the invention, and instructions to use.

Kits may further comprise suitable packaging and/or instructions for use of the compositions. The compositions can be in a dry or lyophilized form, in a solution, particularly a sterile solution, or in a gel or cream. The kit may contain a device for administration or for dispensing the compositions, including, but not limited to, syringe, pipette, transdermal patch and/or microneedle.

The kits may include other therapeutic compounds for use in conjunction with the compounds described herein. These compounds can be provided in a separate form or mixed with the compounds of the present invention.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the invention, kits for treating a subject who suffers from or is susceptible to the conditions described herein are provided, comprising a container comprising a dosage amount of a composition as disclosed herein, and instructions for use. The container can be any of those known in the art and appropriate for storage and delivery.

Kits may also be provided that contain sufficient dosages of the effective composition or compound to provide effective treatment for a subject for an extended period, such as a week, 2 weeks, 3, weeks, 4 weeks, 6 weeks, or 8 weeks or more.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### H4N-Terminal Tails are Required for Nucleosome Binding of G9a and HDAC1

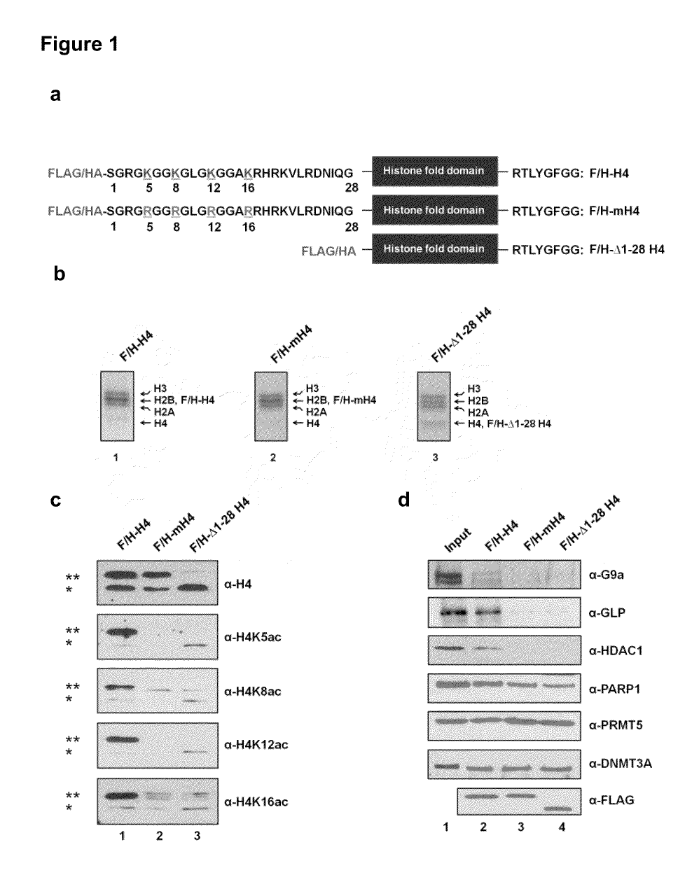

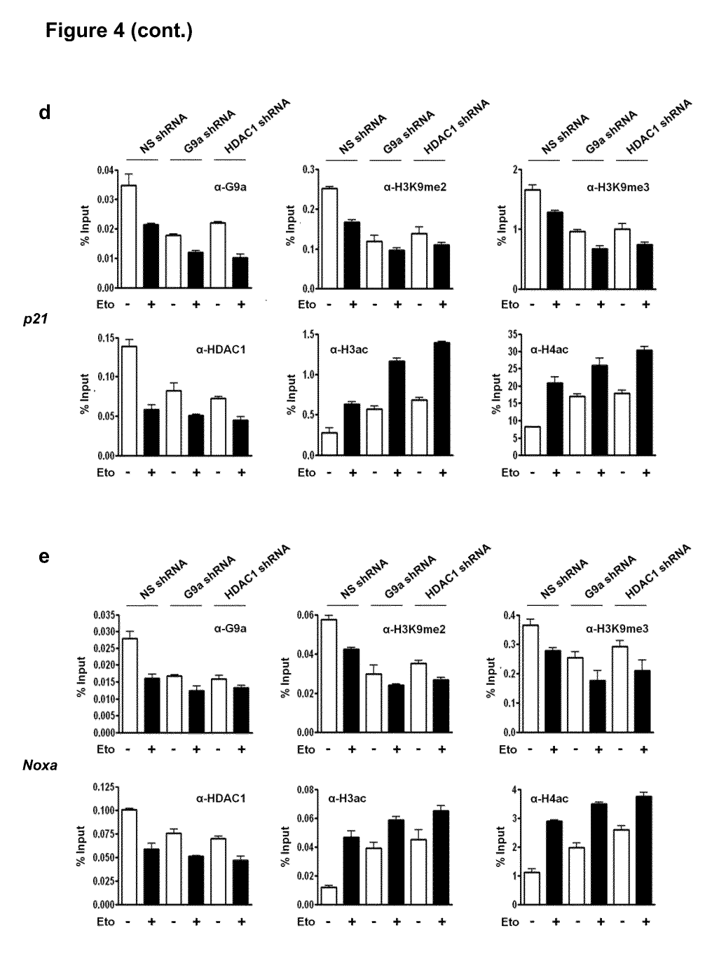

Among various modifications, H4 acetylation has been implicated as a critical mark for activation of p53 target genes upon DNA damage. Since the recognition of acetylated H4 tail protruding from the nucleosome by regulatory factors constitutes a major control point during cellular transcription, we decided to identify proteins that selectively interact with H4-acetylated nucleosomes. Accordingly, we expressed FLAG-HA (F/H)-tagged wild type H4, mutant H4 carrying arginine substitutions at four major acetylation sites (K5, K8, K12 and K16) or tailless H4 lacking the first 28 amino acids in 293T cells (FIG. 1A). Soluble chromatin was prepared from the transfected cells and digested with micrococcal nuclease (MNase) to yield mainly mononucleosomes, as confirmed by gel electrophoresis of nucleosomal DNA (Supplementary FIG. 7). F/H-H4 mononucleosomes and their bound proteins were purified by double immunoprecipitations with anti-FLAG and anti-HA antibodies. Coomassie blue staining confirmed stoichiometry of H2A, H2B, H3 and endogenous/ectopic H4 in the purified nucleosomes (FIG. 1B). In checking the acetylation status of nucleosomal H4 by Western blotting, we detected high levels of wild type ectopic H4 acetylation, but much lower levels of endogenous H4 acetylation (FIG. 1C, lane 1, α-K5/K8/K12/K16ac). As expected, mononucleosomes containing tailless or mutant H4 completely abolished ectopic H4 acetylation (lanes 2 and 3, α-K5/K8/K12/K16ac). The proteomic analysis of the purified materials by multidimensional protein identification technology (MudPIT) revealed that nucleosomes containing wild type, mutant or tailless H4 interact with similar sets of proteins (Supplementary Table S1). Somewhat surprisingly, however, the analysis led us to discover that several proteins including G9a, GLP and HDAC 1 interact with wild type H4 nucleosomes, but not with mutant or tailless H4 nucleosomes (FIG. 1D). These results indicate that the nucleosomal binding of G9a, GLP and HDAC 1 depends on their ability to recognize H4 acetylation within the nucleosome.

### H4 Tail Peptides Penetrate Cells and Stimulate p53 Transactivation

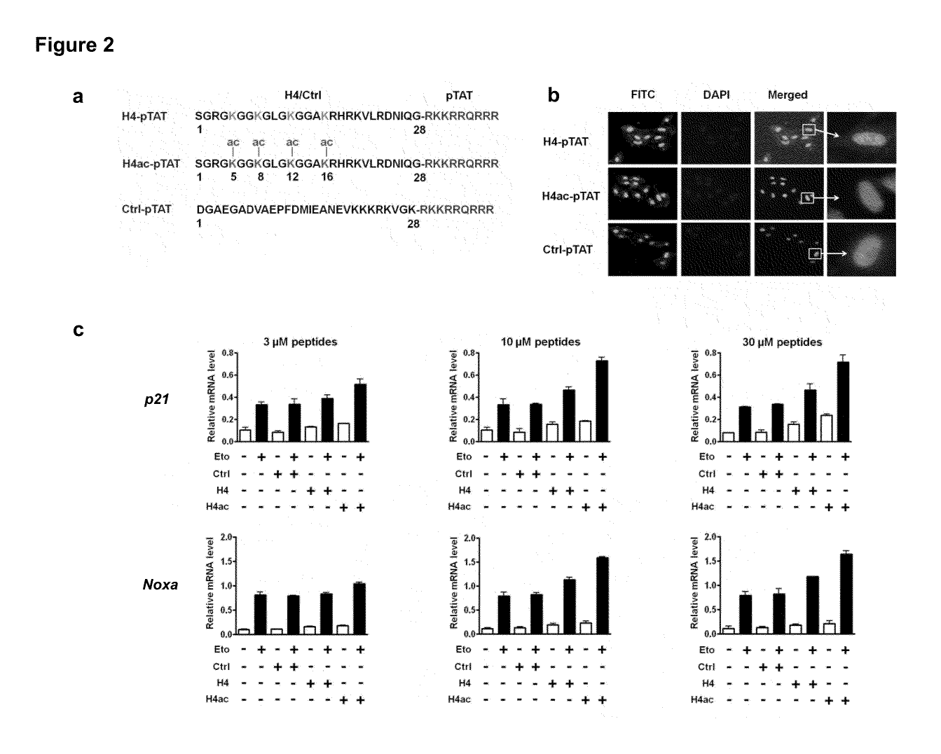

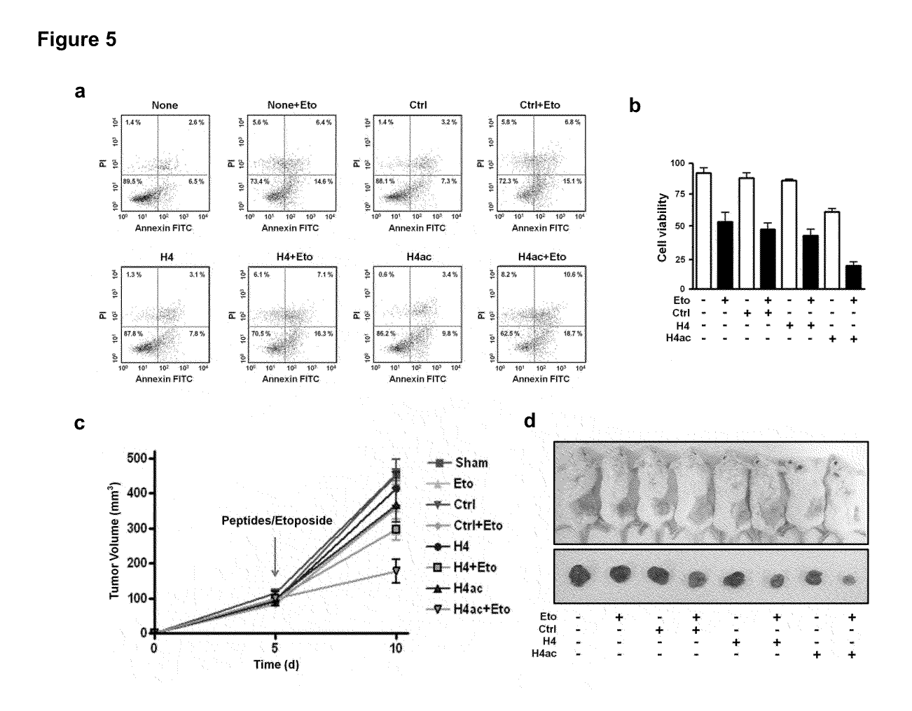

The selective bindings of two chromatin repressors, G9a and HDAC1, to wild type/acetylated H4 nucleosomes imply that acetylated H4 tail peptides could be used as a potent competitor for G9a and HDAC 1. In an attempt to explore this possibility, Applicant synthesized H4 tail fusion peptides comprising the first 28 amino acids of human H4 and the protein transduction domain of HIV TAT (FIG. 2A). To study the effects of acetylation on the tail peptide properties, we prepared a set of H4 tail peptides that were either unmodified (H4-pTAT) or acetylated at the four major lysine substrates (K5, K8, K12 and K16) (H4ac-pTAT). For control reaction, a nonspecific peptide fused to pTAT (Ctrl-pTAT) was also synthesized. Because acetylation is known to increase the secondary structure of H4, especially α-helical content of the tail domain, we performed the circular dichroism (CD) analysis of the synthesized peptides. As summarized in FIG. 8, the CD spectra of unmodified and acetylated tail peptides are dominated by a large negative peak at 200 nm, which is a distinctive feature of disordered proteins. Furthermore, the lack of a shoulder in the range 210-230 nm indicates that both unmodified and acetylated tail peptides have practically no secondary structural elements.

The major drawback in using pTAT-conjugated peptides is their entrapment in cytoplasmic compartments, resulting in limited availability of the peptides in the nucleus. However, H4 tail peptides contain nuclear localization signal (NLS) comprising amino acids 1-21, which allows the nuclear import of the peptides after pTAT-mediated cell penetration (26). Indeed, when human osteosarcoma U2OS cells were incubated with fluorescein isothiocyanate (FITC)-labeled H4 tail peptides for 12 hours, the tail peptides efficiently penetrated cells and preferentially localized in the nucleus (FIG. 2B). Importantly, acetylated and unmodified H4 tail peptides showed comparable abilities to internalize into the nucleus, as similar fluorescence intensity was detected in cell nuclei after their transfection. The control peptides containing typical NLS (KKKRK (SEQ ID NO: 4)) also showed a preferential localization toward the nucleus (FIG. 2B), validating its utility for control experiments.

To examine whether cell permeable H4 tail peptides enhance p53 transactivation, we exposed U2OS cells to three different concentrations (3 μM, 10 μM and 30 μM) of unacetylated, acetylated or control peptides for 12 h, and measured the level of DNA damage-induced transcription of p21 and Noxa genes. Congruent to previously published data (9), Applicant's quantitative reverse transcription PCR (qRT-PCR) showed a sharp increase in p21 transcription in etoposide-treated cells. In checking the effects of H4 tail peptides on p21 transcription, Applicant found that treatment of U2OS cells with 10 μM or 30 μM acetylated H4 tail peptides after etoposide-induced DNA damage stimulates p21 gene transcription (FIG. 2C, upper panel). In contrast, only a modest enhancement of DNA damage-induced p21 transcription was observed in U2OS cells treated with 3 μM acetylated H4 peptides. Moreover, anti-repressive capacity of unacetylated tail peptides was rather moderate in all three concentrations that we tried under DNA damage conditions, indicating that the observed activation of p21 gene transcription by acetylated H4 tail peptides is mainly contributed by acetylation marks in the tail moiety. Under normal conditions, both unmodified and acetylated H4 tail peptides displayed detectable, but limited, capacity to enhance p21 transcription. In exploring the possible effects of H4 tail peptides on other p53 target genes, we also observed a distinct enhancement of DNA damage-induced transcription of Noxa gene after the cellular uptake of H4 tail peptides, especially the acetylated tail peptides (FIG. 2C, lower panel). These results illustrate that the anti-repressive effects of acetylated H4 tail peptides pertain to other p53 target genes as well.

### H4 Tail Peptides Decrease the Occupancy of p53 Target Promoters by G9a and HDAC1

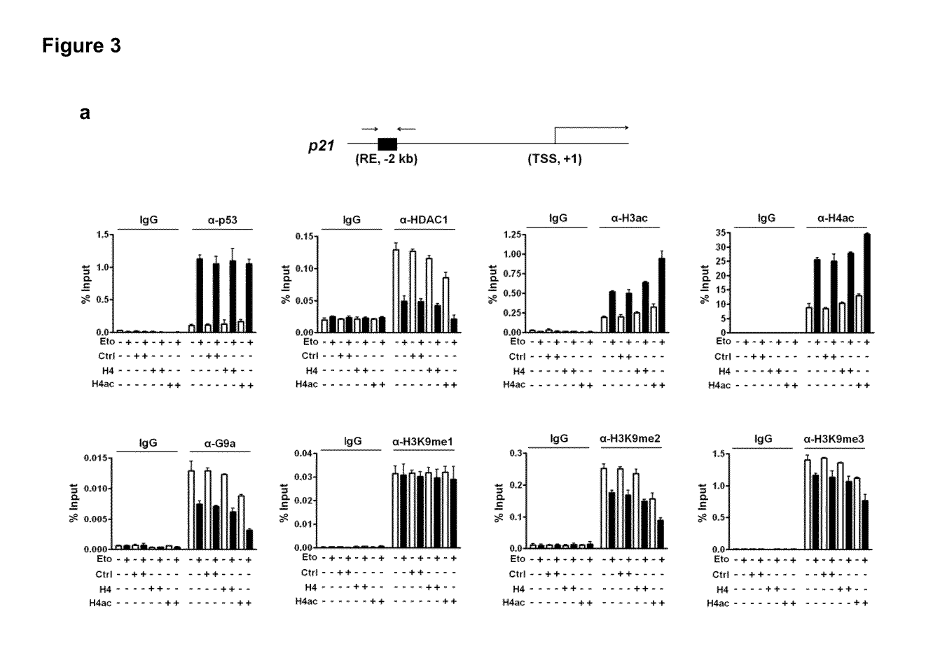

Because G9a and HDAC1 are critical for the maintenance of repressed states of p53 target genes, we next checked whether H4 tail peptides influence the localization of G9a and HDAC1 at p53-responsive promoters by ChIP analysis. As expected, etoposide-induced DNA damage in U2OS cells resulted in distinct accumulations of p53 and H3/H4 acetylation, but generated near complete dissociation of HDAC 1, at the p21 promoter (FIG. 3A, upper panel). When ChIP experiments were repeated with acetylated tail peptide-treated cells, we detected circa 60% more decrease in promoter localization of HDAC 1 and a resultant increase in H3/H4 acetylation (FIG. 3A, upper panel). On the contrary, treating cells with the unmodified tail peptides produced no or relatively minor alterations in HDAC1 occupancy and H3/H4 acetylation at the promoter region (upper panel). In the case of G9a and H3K9 methylation, ChIP experiments reproducibly showed the obvious reduction in G9a and H3K9 di/trimethylation at the p21 promoter after treating cells with acetylated tail peptides (FIG. 3A, lower panel). However, in agreement with the previous indications that G9a is mainly responsible for H3K9 di/trimethylation, the same treatment resulted in no detectable change in H3K9 monomethylation. Notably, anti-repressive action of the tail peptides exhibited acetylation dependence, since unacetylated H4 tail peptides reproducibly showed minimal effects. Both unmodified and acetylated H4 tail peptides generated only limited alterations in the levels of p53, HDAC1, G9a, H3/H4 acetylation and H3K9 di/trimethylation under undamaged conditions (upper panel). To explore the possible effects of H4 tail peptides on other p53 target genes, analogous experiments were carried out on Noxa gene (FIG. 3B). Consistent with the results from p21 gene, acetylated H4 tail peptides, but not unmodified peptides, interfered with the promoter occupancy of G9a and HDAC1. The observed inhibition coincides with a stable reduction of H3-K9 methylation and an apparent increase in H3/H4 acetylation.

To further characterize the anti-repressive action of H4 tail peptides, we examined tail peptide interactions with FLAG-G9a and GST-HDAC1. When the acetylated tail peptides were tested in binding experiments, they showed a direct interaction with G9a and HDAC1, as confirmed by Western blotting with anti-TAT antibody (FIG. 3C, lanes 6 and 12). In contrast, parallel binding experiments with the unmodified tail peptides showed no detectable binding to G9a and minimal binding to HDAC1 (lanes 4 and 10). Thus, consistent with our observation that H4-acetylated nucleosomes preferentially interact with G9a and HDAC 1 (FIG. 1D), these results point to the requirement of acetylation mark for the interaction between H4 tail peptide and G9a/HDAC 1. To the best of our knowledge, this is the first report demonstrating acetylation-dependent binding of G9a/HDAC 1 to H4 tails.

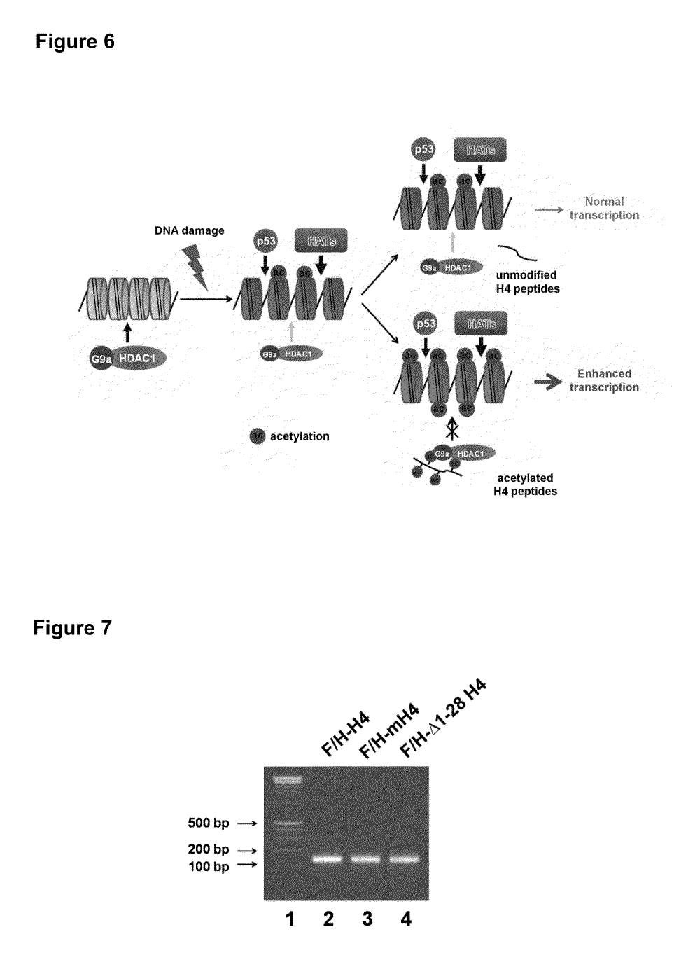

Since G9a and HDAC 1 are known to modify and regulate p53 activity upon DNA damage, we also asked whether H4 tail peptides modulate DNA damage-induced acetylation and methylation of p53. However, treating cells with H4 tail peptides had little or no effect on p53 acetylation at K320, K373 and K382 and p53 methylation at K373 (FIG. 9), indicating that the observed changes in p53-mediated transactivation may not be attributable to p53 modification inhibition. These results constitute a powerful argument that other modifying factors may also play a role in regulating p53 methylation and deacetylation at these sites.

### Stimulatory Effects of H4 Tail Peptides Require G9a and HDAC1

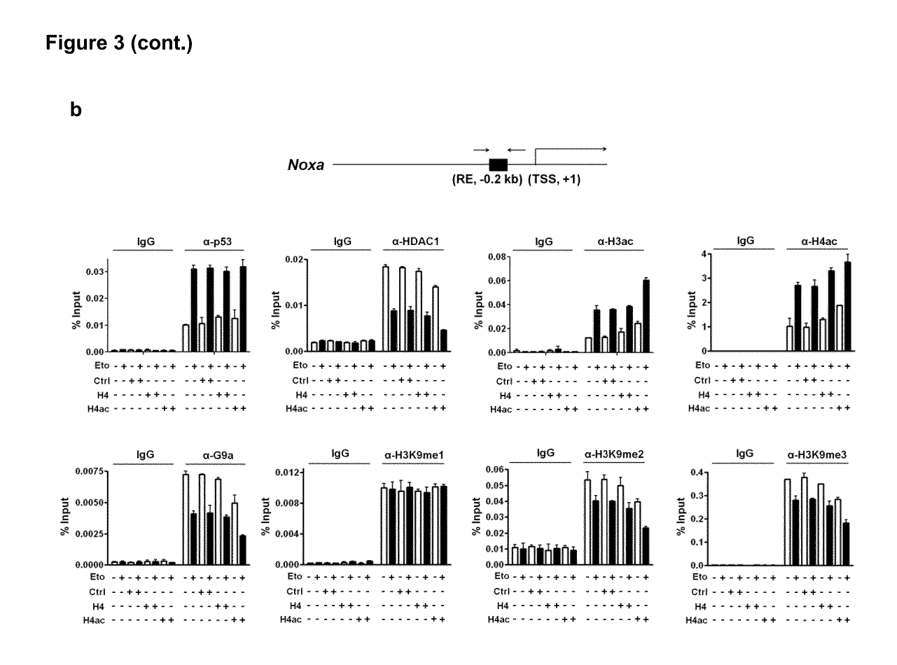

Because p53 target genes could be repressed by cellular factors other than G9a and HDAC1, Applicant further sought to investigate whether H4 tail peptides solely target G9a and HDAC 1 for their stimulatory effects. Accordingly, Applicant stably depleted G9a and HDAC 1 in U2OS cells by employing lentiviral shRNA vectors and checked the impact of H4 tail peptides on p21 and Noxa transcription (FIG. 4A, lanes 2 and 3). For control reactions, cells were also transduced by lentivirus expressing mock shRNA (lane 1). In checking the level of etoposide-induced transcription of p21 and Noxa genes in the mock-depleted cells, enhanced transcription was evident after treatment with acetylated H4 tail peptides, but only minor changes in transcription were observed after treatment with unmodified H4 tail peptides (FIGS. 4B and 4C, NS shRNA). When cells depleted of G9a or HDAC1 were exposed to etoposide, the transcription levels of p21 and Noxa genes were much higher in comparison to their transcription levels in mock-depleted cells (G9a shRNA and HDAC 1 shRNA), confirming repressive roles of G9a and HDAC 1 in p21 and Noxa transcription. Importantly, the fact that tail peptide treatments of the depleted cells failed to yield any detectable enhancement of p21 and Noxa transcription under damaged conditions strongly argues that both G9a and HDAC 1 are necessary for the observed action of H4 tail peptides. Similar peptide treatments of G9a/HDAC1-depleted cells under normal conditions produced no obvious change with respect to transcription of p21 and Noxa genes (FIGS. 4B and 4C, G9a shRNA and HDAC1 shRNA).

To gain support for the above results, we next checked whether depleting G9a or HDAC1 is attributable to the observed alterations in H3-K9 methylation and H3/H4 acetylation by ChIP analysis. Expectedly, treating the mock-depleted cells with etoposide led to the dissociation of G9a and HDAC 1 at the p21 and Noxa promoters (FIGS. 4d and 4e, α-G9a and α-HDAC1, NS shRNA). Because the majority of G9a and HDAC1 were already dissociated from p53 target promoters in response to DNA damage, depletion of G9a and HDAC1 had only a minimal impact on their promoter occupancy (α-G9a and α-HDAC1, G9a shRNA and HDAC1 shRNA). When ChIP experiments were repeated using G9a-depleted cells under normal conditions, Applicant detected near complete loss of G9a (α-G9a, G9a shRNA) and H3-K9 methylation (α-H3K9me2 and α-H3K9me3, G9a shRNA) at the promoters. Surprisingly, however, G9a-depleted cells also showed a distinct reduction in promoter occupancy of HDAC1 (α-HDAC1, G9a shRNA) and a concomitant increase in H3/4 acetylation (α-H3ac and α-H4ac, G9a shRNA). This observation strongly suggests that a stable localization of HDAC1 at p53 target promoters is dependent on G9a. Analogously, in ChIP experiments using HDAC1-depleted cells, near complete loss of G9a and H3-K9 methylation was detected (α-G9a, α-H3K9me2 and α-H3K9me3, HDAC1 shRNA), again arguing for the interdependent localization of G9a and HDAC1 at p21 and Noxa promoters.

### H4 Tail Peptides Sensitize Cells to DNA-Damage Induced Apoptosis

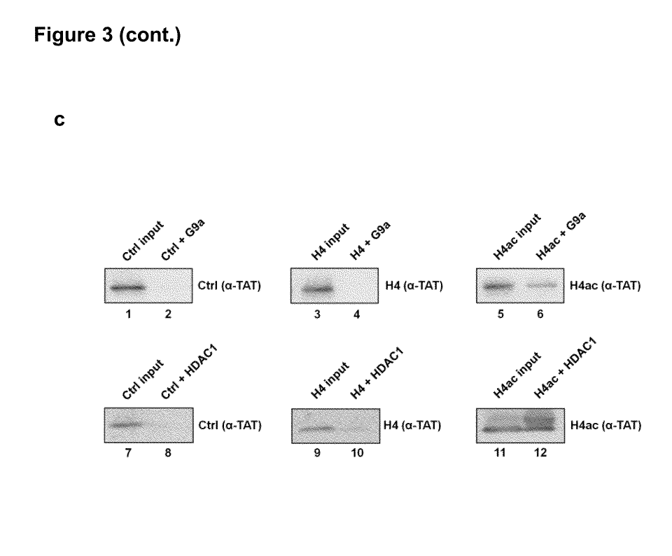

Having established that cellular transduction of acetylated H4 tail peptides establishes active chromatin environment and ameliorates p53 transactivation, we next wanted to know whether cell viability and apoptosis could be altered by H4 tail peptides in U2OS cells. In checking their effects on DNA damage-induced apoptosis, we found that cellular uptake of the acetylated tail peptides significantly increased the fraction of early and late apoptotic cell populations after etoposide exposure (FIG. 5A, None+Eto versus H4ac+Eto). The inability of the unmodified tail peptides to generate similar levels of apoptosis supported the view that acetylation marks are required for the tail peptides to facilitate p53-mediated apoptosis (None+Eto versus H4+Eto). In accordance with the fact that p53 exists in an inactive state and is maintained at low levels under normal conditions, H4 tail peptides produced only minimal apoptotic alterations in undamaged cells (None versus H4 or H4ac). To further study the effects of H4 tail peptides on cell growth, we measured the viability of normal and damaged cells after treating with H4 tail peptides. As summarized in FIG. 5B, acetylated peptide-treated cells showed a significant decrease when compared with unmodified peptide-treated cells under damaged conditions. In contrast, unmodified peptides failed to decrease the rate of cell viability under normal conditions. Interestingly, the peptide transduction also displayed detectably reduced cell viability under normal conditions, reflecting that cellular reactions beside p53 transactivation might be partially affected.

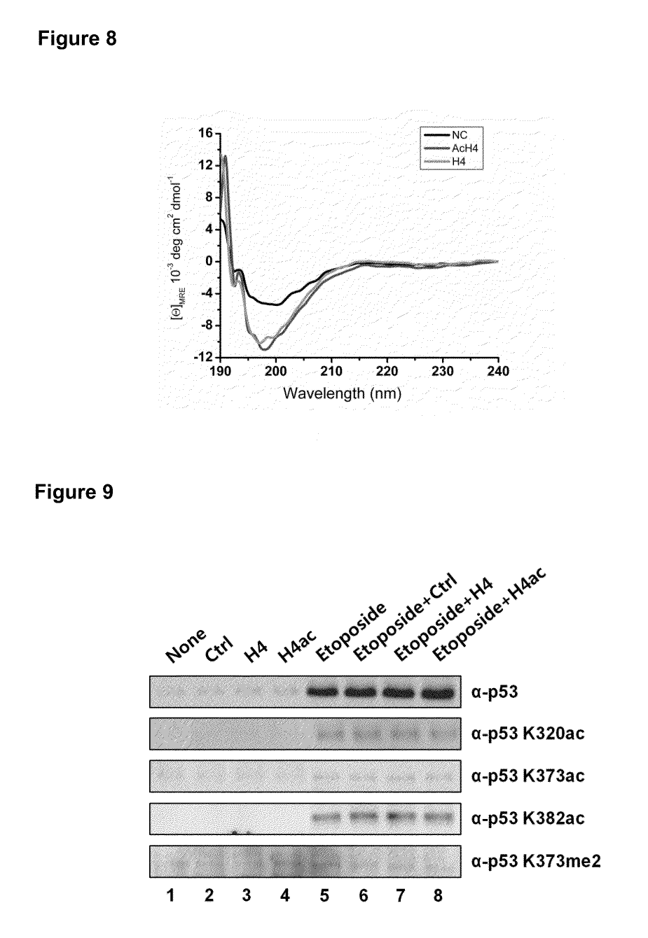

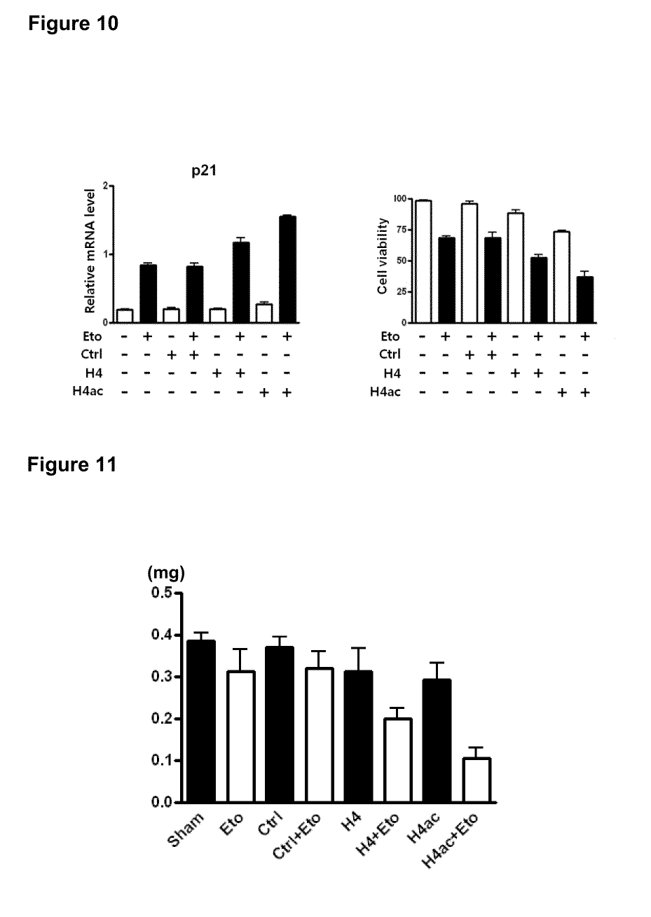

To further validate cell viability and apoptosis results above, the ability of the tail peptides to suppress the growth of cancer cell xenografts was assessed. Since U2OS cells inoculated into mice were inefficient in developing xenograft tumors (data not shown), we employed mouse CT26 colon carcinoma cells for these assays. Of note, the first 28 amino acid sequences are identical in mouse and human histone H4. This extreme conservation of H4 amino acid sequence is consistent with the finding that transfecting CT26 cells with the acetylated tail peptides generates apparent alterations in cell viability and p21 transcription (FIG. 10). BALB/c female mice bearing CT26 colon carcinoma xenograft were randomized into groups and injected with 20 μg of H4 tail peptides over five days. As expected, injection with control peptides did not adversely affect tumor volume and caused no detectable changes in tumor size (FIGS. 5C and 5D). Importantly, however, injection with acetylated H4 tail peptides inhibited tumor growth by 70%, compared with control peptides, while the unacetylated tail peptides inhibited tumor growth only by 40%, at day 10. Note that the anti-tumor growth effects of the acetylated tail peptides were about 2 times more potent than the unmodified tail peptides as assessed by tumor size (FIG. 5C) and weight at day 10 (FIG. 11). Collectively, these data demonstrate that H4 tail peptides have significant in vivo activity in colon cell carcinoma tumor models.

### Discussion

p53 is a key regulator of the DNA damage response by promoting transcription of number of target genes, whose products induce apoptosis and interfere with cell growth. The need to generate new tools to potentiate p53 transcription pathway in cancer cells is clearly illustrated by the current limitations of transcription-independent approaches for triggering p53-deriven apoptosis (25). Despite our lack of understanding of what the endogenous processes might be, histone acetylation is the most characterized and continues to receive the most attention among chromatin remodeling events for p53 transactivation. In our purification of nucleosome-interacting proteins, Applicant identified H4 acetylation as an important mark for the nucleosome-binding by G9a and HDAC1, which are the key repressors of p53-mediated transactivation. This finding led us to hypothesize that acetylated H4 tail peptides could be used as a tool to interfere with G9a and HDAC1 activities, thereby enhancing p53-mediated transactivation. The current study supports this hypothesis by showing that pTAT-mediated delivery of acetylated H4 tail peptides into the cell nucleus stimulates p53 target gene transcription and apoptosis under damaged conditions.

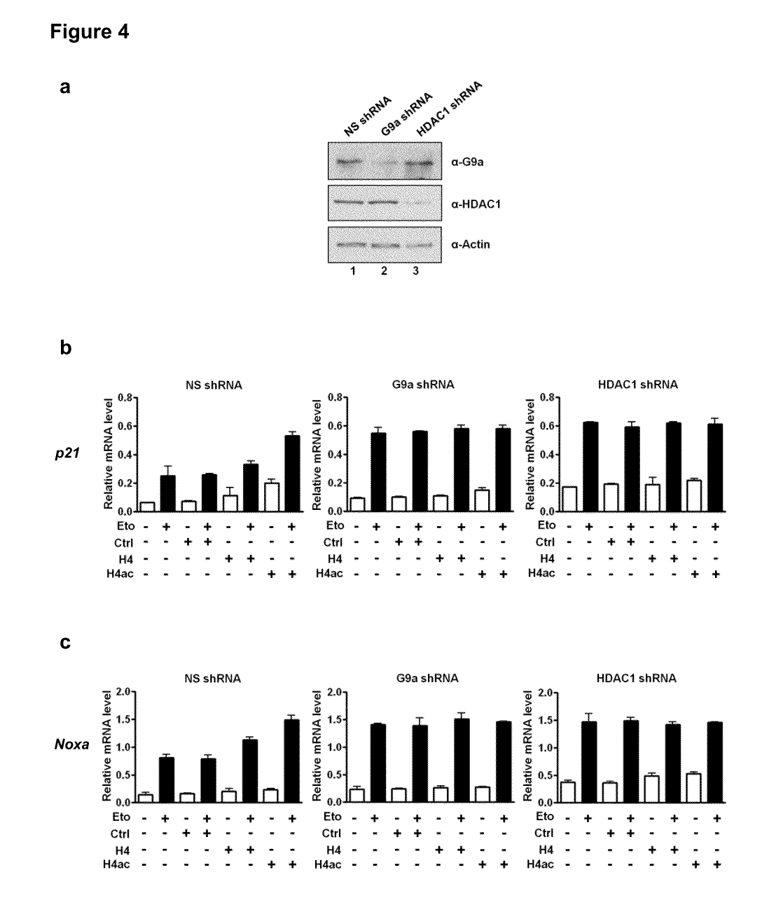

One important observation is that pTAT-fused acetylated H4 tail peptides are able to move into the cell by HIV TAT nuclear penetrating sequence (pTAT) and translocate across the nuclear membrane by nuclear localization signal (NLS, amino acids 1-21) within the H4 tail domain (30). This nuclear-targeted delivery of the tail peptides severely impeded the repressive action of G9a and HDAC 1 and concomitantly augmented p53-mediated transactivation in response to DNA damage. On the other hand, unmodified H4 tail peptides minimally interfered with G9a and HDAC 1 activities and thus only moderately enhanced p53-mediated transcription. The failure of control peptides to have positive effects on p53 transactivation further confirmed that the observed action of H4 tail peptides is accurately measured and correctly interpreted. Importantly, that the acetylated tail peptides trigger near complete loss of G9a and HDAC 1 at p53 target promoters supports our conclusion that the stimulatory effect of the tail peptides is accomplished by their antagonistic action towards G9a and HDAC 1 (FIG. 6). Also, consistent with the observed action of the acetylated tail peptides on p53 transcription pathway, treating cancer cell lines and xenograft models with the acetylated tail peptides, but not with the unmodified tail peptides, led to a sharp decline in tumor cell growth.

A recent study employed similar tail peptide approaches to regulate the interaction between double bromodomain-containing protein 4 (Brd4) and acetylated H4 tails (26). Treatments of P19 and NIH323 cells with acetylated H4 peptides inhibited Brd4-chromatin interactions and reduced the proliferative potential of cells. Our data do not allow us to estimate the potential impact of H4 tail peptides on Brd4 function. However, the finding that BRD4 depletion has little effect on p21 and Noxa transcription in U2OS cells (data not shown) suggests that the observed enhancement of p53 transactivation by the acetylated tail peptides is mainly through the repression of G9a and HDAC 1. Molecular basis of how H4 tail peptides exert the specific effects on different chromatin remodeling factors remains to be elucidated. Unlike other known histone tail-binding proteins, HDAC 1 and G9a do not have any conserved motif such as bromodomain, which is a protein motif to recognize acetylated histone tails. Thus, further studies are necessary to determine the domains of G9a and HDAC 1 required for association with nucleosomes. Nevertheless, the histone tail peptide technology might allow us to define functional contributions of HDAC 1 and G9a in specific cellular events and identify their downstream target genes.

Also of note, recent studies have shown that specific peptides constituting part of a known interface in protein-protein interactions can be used for competition purpose to inhibit target proteins (20, 27, 28, 31). However, the application unique to our method is the utilization of acetylation marks to potentiate the efficacy of the tail peptides in antagonizing HDAC 1 and G9a. Moreover, because HDAC1 and G9a are overexpressed in tumor cells and extend tumor cell life span (14, 32), Applicant's H4 tail peptides are promising drug candidates to molecularly target cancer cells. Although Applicant has mainly focused on acetylated H4 tail peptides, the present study could serve as an important starting point for a broader characterization of properties shared by peptide mimics derived from other histone tails and modifications. It is tempting to speculate that the exposure of cells to other histone tail peptides might have distinct outcomes, via competing for specific factors. Therefore, histone tail peptide mimics with a binding ability to specific chromatin factors will be a useful tool in selectively modulating cellular transcription events.

The present invention having been described by figures, in summary, and in detail, is illustrated and not limited by the following example that includes within itself a number of exemplary, non-limiting compositions and methods.

### Example 1

SEQ ID NO. 1 is SGRGXGGXGL GXGGAXRHRK VLRDNIQG wherein X is independently the same or different and is lysine or a side chain acetylated lysine.

SEQ ID NO. 2 is SGRGXGGXGL GXGGAXRHRK VLRDNIQG wherein X is independently the same or different and is arginine.

SEQ ID NO. 3 is RKKRRQRRR.

### Example 2

### Materials and Methods

### Peptide Synthesis

Peptides were synthesized by standard Fmoc chemistry on an ABI 433A automated peptide synthesizer. Crude peptides were purified by reverse-phase HPLC over C18 preparatory column and confirmed by mass spectrometry. The purity of the synthesized peptides was determined to be greater than 95%. To analyze dose- and time-dependent uptake of the peptides by flow cytometry, aliquots of the peptides were FITC-labeled at its NH2 terminus of the amino acids. Unlabeled peptides were used for all biological assays.

### Cell Lines and Antibodies

The human renal epithelial cell line 293T, human osteosarcoma cell line U2OS and mouse colorectal cancer cell line CT26 were obtained from ATCC and maintained in Dulbecco's Modified Eagle's Medium (DMEM) supplemented with 10% FBS. H4 and G9a antibodies were obtained from Abcam. Antibodies recognizing H3ac, H4ac, H4K5ac, H4K8ac, H4K12ac, H3K9me1 and H3K9me2 were obtained from Millipore. p53 antibody was from Santa Cruz Biotechnology. H3K9me3, H4K16ac and HDAC1 antibodies were from Active Motif. FLAG M2 antibody was from Sigma. TAT antibody was from Cell Application Inc. p53K373/K382ac and p53K320ac antibodies were purchased from Abcam and Millipore, respectively. p53K373me2 antibody was provided by Dr. Huang.

### Nucleosome Purification and Protein Identification

To generate H4 expression plasmids, cDNAs for wild type, lysine-mutated and N-terminal deleted human H4 were subcloned in frame into pIRES vector. For FLAG and HA (F/H) taggings, the coding sequences for the tags were fused to the 5′ end of the H4 cDNAs. Mononucleosomes were prepared as previously described (33). Ectopic H4-containing mononucleosomes and their interacting proteins were isolated from mononucleosome preparations by sequential immunoprecipitation using anti-FLAG M2 and anti-HA antibodies (Sigma) in precipitation buffer (20 mM Tris-pH7.3, 200 mM KCl, 0.2 mM EDTA, 20% Glycerol, and 0.1% NP-40). Proteins co-purified with the F/H-H4 mononucleosomes were identified by multidimensional protein identification technology (MudPIT).

### Circular Dichroism Spectroscopy

Circular dichroism measurements were recorded using a Jasco J-810 spectropolarimeter with a 0.1 cm pathlength cuvette and a peptide concentration of 50 μM. Circular dichroism spectra were obtained at 25° C. in phosphate buffer (10 mM sodium phosphate and 20 mM NaCl, pH 7.4). For each sample, 20 scans from 190 to 250 nm were averaged.

### Quantitative Reverse Transcription PCR

U2OS cells were incubated with medium containing unmodified H4 tail peptides, acetylated H4 tail peptides or control peptides (10 μM each) for 12 h at 37° C. After washing, cells were treated with etoposide (100 μM) for another 12 h and harvested for quantitative reverse transcription PCR (qRT-PCR). Total RNA was prepared using the TRIzol Reagent (Invitrogen) and cDNA was prepared using the iScript cDNA Synthesis kit (Bio-Rad) according to the manufacturer's instructions. Real-time PCR was performed using the IQ SYBR Green Supermix and the IQ5 real time cycler (Bio-Rad). Relative mRNA levels of p21 and Noxa were normalized to β-actin mRNA levels. All reactions were run in triplicate, and the data presented are the average of three individual experiments. Primers for p21 mRNA analysis were: forward, 5′-ATGGAACTTCGACTTTGTCAC-3′ (SEQ ID NO: 5) and reverse 5′-AGGCACAAGGGTACAAGACAGT-3′ (SEQ ID NO: 6) (10). For Noxa, forward 5′-CCAGTTGGAGGCTGAGGTTC-3′ (SEQ ID NO: 7) and reverse 5′-CGTTTCCAAGGGCACCCATG-3′ (SEQ ID NO: 8). For β-actin, forward 5′-GTGGGGCGCCCCAGGCACCA-3′ (SEQ ID NO: 9) and reverse 5′-CTCCTTAATGTCACGCACGATTTC-3′ (SEQ ID NO: 10).

### Chromatin Immunoprecipitation

After treating U2OS cells with peptides and etoposide as in qRT-PCR analysis, ChIP assays were performed as described (5). Primers for the amplification of the p21 promoter were: forward, 5′-TGGACTGGGCACTCTTGTCC-3′ (SEQ ID NO: 11) and reverse, 5′-CAGAGTAACAGGCTAAGGTT-3′ (SEQ ID NO: 12). Primers for the amplification of the Noxa promoter were: forward, 5′-GTCCAGCGTTTGCAGATG-3′ (SEQ ID NO: 13) and reverse 5′-AACGAGGTGGGAGGAGAA-3′ (SEQ ID NO: 14) Annealing temperatures for p21 and Noxa primers were 55° C. and 58° C., respectively. All reactions were run in triplicate, and data presented is the average of three individual experiments.

### RNA Interference.

For shRNA-based knockdown of HDAC1, the target sequence (5′-GC AGATGCAGAGATTCAAC-3′ (SEQ ID NO: 15)) was subcloned into the pSUPER (pS) vector according to the manufacturer's recommendations (Oligoengine). To silence G9a, the target sequence (5′-GACAGCAAGTATGAAGTTAAAGCTC-3′ (SEQ ID NO: 16)) was inserted into the pLKO.1-Puro lentiviral vector which was kindly provided by Dr. M. Stallcup. U2OS cells were transfected with pS-HDAC1 vector by using Lipofectamine (Invitrogen) or G9a lentivirus and selected with puromycin (2 μg/ml) for 7 days.

### Cell Viability and Apoptosis Assays

For cell viability assay, U2OS and CT26 cells were transfected with H4 tail or control peptides for 12 h, and treated with etoposide (100 μM) for another 12 h. After washing cells with PBS, cells were stained with trypan blue and determined the percentage of survived cells. For apoptosis assay, the cells were harvested with trypsin/EDTA, resuspended in the binding buffer and then incubated with FITC-conjugated Annexin V and propidium iodide (BD Pharmingen), according to the manufacturer's protocol. The percentage of apoptotic cells were assessed by flow cytometry.

### Xenograft Tumor Model

All animal experiments were performed according to protocols approved by the institutional animal care and use committee. Tumor xenografts were established by subcutaneous injection of 1×107 CT26 cells into eight-week-old female BALB/c mice (Central Lab Animal Inc). At day 5 post injection, mice bearing CT26 tumor xenografts were treated with H4 tail peptides (20 μg) and etoposide (12 mg/kg) for 5 days. Tumor volumes were measured with calipers at days 5 and 10. All mice were sacrificed by asphyxiation with CO2, and tumors were excised and weighed 10 days after the cell injection.

### Example 3

### Table S1

Table S1 is a summary of proteins copurified with wild type and mutant H4 nucleosomes. H4 Ectopic H4 nucleosomes and their associated factors were purified from nuclear extracts of 293T cells expressing Flag-HA-H4 wild type or mutant through sequential anti-Flag and anti-HA immunoaffinity chromatographies. Three independent affinity purifications were used for the MudPIT mass spectrometry analysis. The table summarizes the peptide count and the amino acid coverage of proteins co-purified with ectopic H4 nucleosomes. Light grey and underlined lettering indicates unique binding proteins.

TABLE S1

| | Wild type H4 | Mutant H4 | (Δ1-28) H4 |
|---|---|---|---|
| | nucleosome | nucleosome | nucleosome |
| Histone proteins | | | |
| Histone H3 | 6 (82.6%) | 6 (80.6%) | 7 (89.6%) |
| Histone H4 | 7 (84.6%) | 7 (81.7%) | 5 (62.7%) |
| Histone H2A | 5 (63.1%) | 6 (72.3%) | 6 (74.1%) |
| Histone H2B | 5 (54.7%) | 5 (55.1%) | 5 (59.4%) |
| Histone H2AX | 2 (13.7%) | 1 (9.1%) | 1 (8.4%) |
| macroH2A | 11 (55.1%) | 10 (49.8%) | 10 (48.2%) |
| H1F0 | 3 (21.2%) | 2 (17.6%) | 2 (16.2%) |
| H1A | 2 (11.2%) | 1 (5.2%) | 2 (11.8%) |
| H1C | 2 (10.3%) | 2 (11.8%) | 2 (12.3%) |
| H1D | 1 (8.6%) | 1 (7.2%) | 1 (6.4%) |
| H1E | 1 (6.2%) | 1 (6.5%) | 2 (11.8%) |
| H1B | 2 (12.5%) | 2 (12.3%) | 2 (12.8%) |
| Chaperones | | | |
| CBX5 (HP1α) | 3 (21.4%) | 3 (22.6%) | 3 (21.4%) |
| CBX1 (HP1β) | 2 (17.3%) | 3 (21.8%) | 3 (22.8%) |
| CBX3 (HP1γ) | 2 (13.2%) | 3 (19.6%) | 2 (14.7%) |
| Spt16 | 12 (17.2%) | 11 (18.5%) | 12 (21.6%) |
| SSRP1 | 9 (15.8%) | 9 (14.7%) | 7 (11.9%) |
| Nucleolin | 6 (12%) | 6 (11.3%) | 8 (16.3%) |
| Chromatin | | | |
| remodeling factors | | | |
| RUVBL1 | 7 (19.1%) | 6 (18.2%) | 8 (21.3%) |
| RUVBL2 | 5 (13.4%) | 6 (14.2%) | 8 (16.3%) |
| CHAF1A | 3 (5.5%) | 1 (1.8%) | 3 (4.9%) |
| RSF1 | 12 12.2%) | 7 (8.1%) | 10 (11.7%) |
| CHD4 | 2 (1.2%) | 1 (0.6%) | 1 (0.6%) |
| MeCP2 | 5 (12.3%) | 1 (2.8%) | 1 (3.1%) |
| SMARCA1 | 11 (24.9%) | 11 (22.6%) | 9 (19.2%) |
| SMARCA5 | 9 (20%) | 10 (22.1%) | 13 (28.3%) |
| BAF170 | 3 (4.3%) | 0 | 0 |
| TAF4 | 5 (6.6%) | 0 | 0 |
| TRAP150 | 2 (3.4%) | 0 | 0 |
| SRCAP | 0 | 0 | 3 (1.9%) |
| Histone and DNA | | | |
| modifying factors | | | |
| PRMT5 | 9 (21.8%) | 7 (17.2%) | 9 (23.8%) |
| DNMT3A | 11 (29%) | 7 (19.5%) | 8 (23.7%) |
| DNMT3B | 12 (31.8%) | 6 (17.1%) | 11 (30.6%) |
| Suz12 | 3 (5.1%) | 1 (1.8%) | 1 (1.4%) |
| EuHMT1 | 3 (5.7%) | 0 | 0 |
| EuHMT2 | 2 (4%) | 0 | 0 |
| HDAC1 | 5 (12.9%) | 0 | 0 |
| DNA repair factors | | | |
| PARP1 | 21 (41.3%) | 18 (38.6%) | 23 (44.2%) |
| TOPO1 | 5 (7.4%) | 3 (4.8%) | 3 (4.2%) |
| DDB1 | 3 (5.4%) | 3 (4.8%) | 2 (3.7%) |
| XRCC6 | 2 (5.7%) | 0 | 1 (3.2%) |
| XRCC5 | 2 (4.4%) | 0 | 2 (5.3%) |
| MDC1 | 0 | 0 | 4 (18.4%) |
| RAD23B | 0 | 0 | 1 (8.9%) |
| Others | | | |
| MTF2 | 2 (4.6%) | 0 | 0 |
| TCF20 | 3 (2.4%) | 0 | 0 |
| Mtf2 | 0 | 0 | 0 |
| Tcf20 | 0 | 0 | 0 |
| P32 | 0 | 3 (21.7%) | 0 |
| P66 | 0 | 6 (14.8%) | 0 |
| SFRS9 | 0 | 1 (8.1%) | 0 |
| ASF1 | 0 | 0 | 2 (18.8%) |
| ARF1 | 0 | 0 | 1 (8.8%) |
| WDR77 | 0 | 0 | 2 (11.4%) |
| DAXX | 0 | 0 | 1 (2.4%) |

### Tables 1A, 1B, and 1C

Tables 1A, 1B and 1C are summaries of nucleosome-interacting proteins identified by mass spectrometry. A mixture of proteins were copurified with nucleosomes containing wild type, lysine mutant or tailless H4, and identified by tandem mass spectrometry analysis. The numbers of peptide hits were listed as tabulated below.

TABLE 1A
Wild type H4 nucleosome

| | Protein | Accession number |
|---|---|---|
| Histone Proteins | | |
| histone H3 | H3 | N/A |
| histone H4 | H4 | N/A |
| histone H2A | H2A | N/A |
| histone H2B | H2B | N/A |
| histone H2AX | H2AFX | GI: 52630339 |
| macroH2A1.1 and macroH2A1.2 | H2AFY | GI: 93141010, |
| | | GI: 20336744 |
| H1 histone family, member 0 (H1F0) | H1.0 | GI: 85838503 |
| histone cluster 1, H1a (HIST1H1A) | H1.1 | GI: 116256359 |
| histone cluster 1, H1c (HIST1H1C) | H1.2 | GI: 21071025 |
| histone cluster 1, H1d (HIST1H1D) | H1.3 | GI: 20544161 |
| histone cluster 1, H1e (HIST1H1E) | H1.4 | GI: 20544164 |
| histone cluster 1, H1b (HIST1H1B) | H1.5 | GI: 15718716 |
| Chaperones | | |
| chromobox homolog 5 (HP1 alpha homolog, | CBX5 | GI: 188035909 |
| Drosophila) | | |
| chromobox homolog 1 (HP1 beta homolog | CBX1 | GI: 187960060 |
| Drosophila) | | |
| chromobox homolog 3 (HP1 gamma homolog, | CBX3 | GI: 20544152 |
| Drosophila) | | |
| suppressor of Ty 16 homolog (S. cerevisiae) | Spt16 | GI: 223890260 |
| structure specific recognition protein 1 | SSRP1 | GI: 28416943 |
| high mobility group proteins | HMG | N/A |
| | families | |
| nucleolin | NCL | GI: 55956787 |
| Chromatin remodeling and transcription factors | | |
| RuvB-like 1 (E. coli) | RUVBL1 | GI: 197276633 |
| RuvB-like 2 (E. coli) | RUVBL2 | GI: 14713519 |
| chromatin assembly factor 1, subunit A | CHAF1A | GI: 50513244 |
| remodeling and spacing factor 1 | RSF1 | GI: 38788332 |
| chromodomain helicase DNA binding protein 4 | CHD4 | GI: 24047225 |
| methyl CpG binding protein 2 (Rett syndrome) | MeCP2 | GI: 160707948 |
| SWI/SNF related, matrix associated, actin | SMARCA1 | GI: 164419747 |
| dependent | | |
| regulator of chromatin, subfamily a, member 1 and 5 | SMARCA5 | GI: 21071057 |
| tripartite motif-containing 28 | TRIM28 | GI: 14971416 |
| retinoblastoma binding protein 4 | RBBP4 | GI: 207029375 |
| SWI/SNF complex 170 KDa subunit | BAF170* | GI: 1549240 |
| TATA box binding protein-associated factor, | TAF4* | GI: 110832842 |
| 135 kDa | | |
| thyroid hormone receptor-associated protein | TRAP150* | GI: 4530440 |
| complex component | | |
| Histone and DNA modifying factors | | |
| protein arginine methyltransferase 5 | PRMT5 | GI: 88900506 |
| DNA (cytosine-5-)-methyltransferase 3 alpha | DNMT3A | GI: 28559068 |
| DNA (cytosine-5-)-methyltransferase 3 beta | DNMT3B | GI: 28559059 |
| suppressor of zeste 12 homolog (Drosophila) | Suz12 | GI: 16041674 |
| euchromatic histone-lysine N-methyltransferase 2 | EuHMT2* | GI: 156142198 |
| (G9a) | (G9a) * | |
| histone deacetylase 1 | HDAC1* | GI: 38197109 |
| DNA repair factors | | |
| poly (ADP-ribose) polymerase 1 | PARP1 | GI: 156523967 |
| topoisomerase (DNA) I | TOPO1 | GI: 19913404 |
| damage-specific DNA binding protein 1, 127 kDa | DDB1 | GI: 148529013 |
| X-ray repair complementing defective repair in | XRCC6* | GI: 48735092 |
| Chinese hamster cells 6 | | |
| X-ray repair complementing defective repair in | XRCC5* | GI: 195963391 |
| Chinese hamster cells 5 | | |
| Ribosomal proteins | | |
| heterogeneous nuclear ribonucleoprotein K | HNRNPK | GI: 186659504 |
| heterogeneous nuclear ribonucleoprotein U | HNRNPU | GI: 74136882 |
| Others | | |
| metastasis associated 1 | MTA1 | GI: 1008543 |
| metastasis associated 1 family, member 2 | MTA2 | GI: 14141169 |
| metal response element binding transcription factor 2 | MTF2* | GI: 166706893 |
| transcription factor 20 (AR1) | TCF20* | GI: 31652243 |

TABLE 1B
Mutant H4 nucleosome

| | Protein | Accession number |
|---|---|---|
| Histone Proteins | | |
| histone H3 | H3 | N/A |
| histone H4 | H4 | N/A |
| histone H2A | H2A | N/A |
| histone H2B | H2B | N/A |
| histone H2AX | H2AFX | GI: 52630339 |
| macroH2A1.1 and macroH2A1.2 | H2AFY | GI: 93141010, |
| | | GI: 20336744 |
| H1 histone family, member 0 (H1F0) | H1.0 | GI: 85838503 |
| histone cluster 1, H1a (HIST1H1A) | H1.1 | GI: 116256359 |
| histone cluster 1, H1c (HIST1H1C) | H1.2 | GI: 21071025 |
| histone cluster 1, H1d (HIST1H1D) | H1.3 | GI: 20544161 |
| histone cluster 1, H1e (HIST1H1E) | H1.4 | GI: 20544164 |
| histone cluster 1, H1b (HIST1H1B) | H1.5 | GI: 15718716 |
| Chaperones | | |
| chromobox homolog 5 (HP1 alpha homolog, | CBX5 | GI: 188035909 |
| Drosophila) | | |
| chromobox homolog 1 (HP1 beta homolog | CBX1 | GI: 187960060 |
| Drosophila) | | |
| chromobox homolog 3 (HP1 gamma homolog, | CBX3 | GI: 20544152 |
| Drosophila) | | |
| suppressor of Ty 16 homolog (S. cerevisiae) | Spt16 | GI: 223890260 |
| structure specific recognition protein 1 | SSRP1 | GI: 28416943 |
| high mobility group proteins | HMG | N/A |
| | families | |
| nucleolin | NCL | GI: 55956787 |
| Chromatin remodeling and transcription factors | | |
| RuvB-like 1 (E. coli) | RUVBL1 | GI: 197276633 |
| RuvB-like 2 (E. coli) | RUVBL2 | GI: 14713519 |
| chromatin assembly factor 1, subunit A | CHAF1A | GI: 50513244 |
| remodeling and spacing factor 1 | RSF1 | GI: 38788332 |
| chromodomain helicase DNA binding protein 4 | CHD4 | GI: 24047225 |
| methyl CpG binding protein 2 (Rett syndrome) | MeCP2 | GI: 160707948 |
| SWI/SNF related, matrix associated, actin dependent | SMARCA1 | GI: 164419747 |
| regulator of chromatin, subfamily a, member 1 and 5 | SMARCA5 | GI: 21071057 |
| tripartite motif-containing 28 | TRIM28 | GI: 14971416 |
| retinoblastoma binding protein 4 | RBBP4 | GI: 207029375 |
| Histone and DNA modifying factors | | |
| protein arginine methyltransferase 5 | PRMT5 | GI: 88900506 |
| DNA (cytosine-5-)-methyltransferase 3 alpha | DNMT3A | GI: 28559068 |
| DNA (cytosine-5-)-methyltransferase 3 beta | DNMT3B | GI: 28559059 |
| suppressor of zeste 12 homolog (Drosophila) | Suz12 | GI: 16041674 |
| DNA repair factors | | |
| poly (ADP-ribose) polymerase 1 | PARP1 | GI: 156523967 |
| topoisomerase (DNA) I | TOPO1 | GI: 19913404 |
| damage-specific DNA binding protein 1, 127 kDa | DDB1 | GI: 148529013 |
| X-ray repair complementing defective repair in | XRCC6* | GI: 48735092 |
| Chinese hamster cells 6 | | |
| Ribosomal proteins | | |
| heterogeneous nuclear ribonucleoprotein K | HNRNPK | GI: 186659504 |
| heterogeneous nuclear ribonucleoprotein U | HNRNPU | GI: 74136882 |
| heterogeneous nuclear ribonucleoprotein F | HNRNPF* | GI: 148470396 |
| heterogeneous nuclear ribonucleoprotein C | HNRNPC* | GI: 117189974 |
| Others | | |
| metastasis associated 1 | MTA1 | GI: 1008543 |
| metastasis associated 1 family, member 2 | MTA2 | GI: 14141169 |
| CD8 antigen alpha polypeptide isoform 1 precursor | p32* | GI: 28872801 |
| translation initiation factor eIF3 p66 subunit | p66* | GI: 2351377 |
| splicing factor, arginine/serine-rich 9 | SFRS9* | GI: 38016912 |

TABLE 1C
(Δ1-28) H4 nucleosome

| | Protein | Accession number |
|---|---|---|
| Histone Proteins | | |
| histone H3 | H3 | N/A |
| histone H4 | H4 | N/A |
| histone H2A | H2A | N/A |
| histone H2B | H2B | N/A |
| histone H2AX | H2AFX | GI: 52630339 |
| macroH2A1.1 and macroH2A1.2 | H2AFY | GI: 93141010, |
| | | GI: 20336744 |
| H1 histone family, member 0 (H1F0) | H1.0 | GI: 85838503 |
| histone cluster 1, H1a (HIST1H1A) | H1.1 | GI: 116256359 |
| histone cluster 1, H1c (HIST1H1C) | H1.2 | GI: 21071025 |
| histone cluster 1, H1d (HIST1H1D) | H1.3 | GI: 20544161 |
| histone cluster 1, H1e (HIST1H1E) | H1.4 | GI: 20544164 |
| histone cluster 1, H1b (HIST1H1B) | H1.5 | GI: 15718716 |
| Chaperones | | |
| chromobox homolog 5 (HP1 alpha homolog, | CBX5 | GI: 188035909 |
| Drosophila) | | |
| chromobox homolog 1 (HP1 beta homolog | CBX1 | GI: 187960060 |
| Drosophila) | | |
| chromobox homolog 3 (HP1 gamma homolog, | CBX3 | GI: 20544152 |
| Drosophila) | | |
| suppressor of Ty 16 homolog (S. cerevisiae) | Spt16 | GI: 223890260 |
| structure specific recognition protein 1 | SSRP1 | GI: 28416943 |
| high mobility group proteins | HMG | N/A |
| | families | |
| nucleolin | NCL | GI: 55956787 |
| Chromatin remodeling and transcription factors | | |
| RuvB-like 1 (E. coli) | RUVBL1 | GI: 197276633 |
| RuvB-like 2 (E. coli) | RUVBL2 | GI: 14713519 |
| chromatin assembly factor 1, subunit A | CHAF1A | GI: 50513244 |
| remodeling and spacing factor 1 | RSF1 | GI: 38788332 |
| chromodomain helicase DNA binding protein 4 | CHD4 | GI: 24047225 |
| methyl CpG binding protein 2 (Rett syndrome) | MeCP2 | GI: 160707948 |
| SWI/SNF related, matrix associated, actin dependent | SMARCA1 | GI: 164419747 |
| regulator of chromatin, subfamily a, member 1 and 5 | SMARCA5 | GI: 21071057 |
| tripartite motif-containing 28 | TRIM28 | GI: 14971416 |
| retinoblastoma binding protein 4 | RBBP4 | GI: 207029375 |
| Snf2-related CREBBP activator protein | SRCAP* | GI: 166796214 |
| Histone and DNA modifying factors | | |
| protein arginine methyltransferase 5 | PRMT5 | GI: 88900506 |
| DNA (cytosine-5-)-methyltransferase 3 alpha | DNMT3A | GI: 28559068 |
| DNA (cytosine-5-)-methyltransferase 3 beta | DNMT3B | GI: 28559059 |
| suppressor of zeste 12 homolog (Drosophila) | Suz12 | GI: 16041674 |
| DNA repair factors | | |
| poly (ADP-ribose) polymerase 1 | PARP1 | GI: 156523967 |
| topoisomerase (DNA) I | TOPO1 | GI: 19913404 |
| damage-specific DNA binding protein 1, 127 kDa | DDB1 | GI: 148529013 |
| mediator of DNA-damage checkpoint 1 | MDC1* | GI: 132626687 |
| RAD23 homolog B (S. cerevisiae) | RAD23B* | GI: 51173731 |
| Ribosomal proteins | | |
| heterogeneous nuclear ribonucleoprotein K | HNRNPK | GI: 186659504 |
| heterogeneous nuclear ribonucleoprotein U | HNRNPU | GI: 74136882 |
| heterogeneous nuclear ribonucleoprotein F | HNRNPF* | GI: 148470396 |
| heterogeneous nuclear ribonucleoprotein C | HNRNPC* | GI: 117189974 |
| heterogeneous nuclear ribonucleoprotein H1 | HNRNPH1* | GI: 186287258 |
| Others | | |
| metastasis associated 1 | MTA1 | GI: 1008543 |
| metastasis associated 1 family, member 2 | MTA2 | GI: 14141169 |
| anti-silencing function 1 homolog B | ASF1* | GI: 33874941 |
| ADP-ribosylation factor 1 | ARF1* | GI: 20147654 |
| WD repeat domain 77 | WDR77* | GI: 20127622 |
| death-domain associated protein | DAXX* | GI: 215422387 |

*indicates unique binding protein

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

1. Harris S L & Levine A J (2005) The p53 pathway: positive and negative feedback loops. Oncogene, 24:2899-2908.
2. Kruse J P & Gu W (2009) Modes of p53 regulation. Cell, 137:609-622.
3. Laptenko 0 & Prives C (2006) Transcriptional regulation by p53: one protein, many possibilities. Cell Death Differ, 13:951-961.
4. Vousden K H & Lane D P (2007) p53 in health and disease. Nat Rev Mol Cell Biol, 8:275-283.
5. An W, Kim J, & Roeder R G (2004) Ordered cooperative functions of PRMT1, p300, and CARM1 in transcriptional activation by p53. Cell, 117:735-748.
6. Barlev N A, et al. (2001) Acetylation of p53 activates transcription through recruitment of coactivators/histone acetyltransferases. Mol Cell, 8:1243-1254.
7. Bochar D A, et al. (2000) BRCA1 is associated with a human SWI/SNF-related complex: linking chromatin remodeling to breast cancer. Cell, 102:257-265.
8. Espinosa J M & Emerson B M (2001) Transcriptional regulation by p53 through intrinsic DNA/chromatin binding and site-directed cofactor recruitment. Mol Cell, 8:57-69.
9. Haberland M, Montgomery R L, & Olson E N (2009) The many roles of histone deacetylases in development and physiology: implications for disease and therapy. Nat Rev Genet, 10:32-42.
10. Miremadi A, Oestergaard M Z, Pharoah P D, & Caldas C (2007) Cancer genetics of epigenetic genes. Hum Mol Genet, 16 Spec No 1:R28-49.
11. Muntean A G & Hess J L (2009) Epigenetic dysregulation in cancer. Am J Pathol, 175:1353-1361.
12. Spannhoff A, Hauser A T, Heinke R, Sippl W, & Jung M (2009) The emerging therapeutic potential of histone methyltransferase and demethylase inhibitors. ChemMedChem, 4:1568-1582.
13. Spotswood H T & Turner B M (2002) An increasingly complex code. J Clin Invest, 110:577-582.
14. Stimson L, Wood V, Khan O, Fotheringham S, & La Thangue N B (2009) HDAC inhibitor-based therapies and haematological malignancy. Ann Oncol, 20:1293-1302.
15. Tan J, et al. (2007) Pharmacologic disruption of Polycomb-repressive complex 2-mediated gene repression selectively induces apoptosis in cancer cells. Genes Dev, 21:1050-1063.
16. Kim J K, Esteve P O, Jacobsen S E, & Pradhan S (2009) UHRF1 binds G9a and participates in p21 transcriptional regulation in mammalian cells. Nucleic Acids Res, 37:493-505.
17. Peterson C L & Laniel M A (2004) Histones and histone modifications. Curr Biol, 14:R546-551.
18. Issaeva N, et al. (2003) Rescue of mutants of the tumor suppressor p53 in cancer cells by a designed peptide. Proc Natl Acad Sci USA, 100:13303-13307.
19. Joliot A & Prochiantz A (2004) Transduction peptides: from technology to physiology. Nat Cell Biol, 6:189-196.
20. Moellering R E, et al. (2009) Direct inhibition of the NOTCH transcription factor complex. Nature, 462:182-188.
21. Polo J M, et al. (2004) Specific peptide interference reveals BCL6 transcriptional and oncogenic mechanisms in B-cell lymphoma cells. Nat Med, 10:1329-1335.
22. Snyder E L, Meade B R, Saenz C C, & Dowdy S F (2004) Treatment of terminal peritoneal carcinomatosis by a transducible p53-activating peptide. PLoS Biol, 2:E36.
23. Frankel A D, Bredt D S, & Pabo C O (1988) Tat protein from human immunodeficiency virus forms a metal-linked dimer. Science, 240:70-73.
24. Wadia J S & Dowdy S F (2005) Transmembrane delivery of protein and peptide drugs by TAT-mediated transduction in the treatment of cancer. Adv Drug Deliv Rev, 57:579-596.
25. Moll U M, Wolff S, Speidel D, & Deppert W (2005) Transcription-independent pro-apoptotic functions of p53. Curr Opin Cell Biol, 17:631-636.
26. Nishiyama A, et al. (2008) Intracellular delivery of acetyl-histone peptides inhibits native bromodomain-chromatin interactions and impairs mitotic progression. FEES Lett, 582:1501-1507.
27. Souroujon M C & Mochly-Rosen D (1998) Peptide modulators of protein-protein interactions in intracellular signaling. Nat Biotechnol, 16:919-924.
28. Walensky L D, et al. (2004) Activation of apoptosis in vivo by a hydrocarbon-stapled BH3 helix. Science, 305:1466-1470.
29. Espinosa J M, Verdun R E, and Emerson B M. p53 functions through stress- and promoter-specific recruitment of transcription initiation components before and after DNA damage. Mol Cell 2003; 12: 1015-1027.
30. Mosammaparast N, Guo Y, Shabanowitz J, Hunt D F, Pemberton L F. Pathways mediating the nuclear import of histones H3 and H4 in yeast. J Biol Chem 2002; 277: 862-868.
31. Harada Y, Kanehira M, Fujisawa Y, Takata R, Shuin T, Miki T, et al. Cell-permeable peptide DEPDC1-ZNF224 interferes with transcriptional repression and oncogenicity in bladder cancer cells. Cancer Res 2010; 70: 5829-5839.
32. Huang J, Dorsey J, Chuikov S, Pérez-Burgos L, Zhang X, Jenuwein T, et al. G9a and Glp methylate lysine 373 in the tumor suppressor p53. J Biol Chem 2010; 285: 9636-9641.
33. Heo K, Kim H, Choi S H, Kim K, Gu J, Lieber M R et al. FACT-mediated exchange of histone variant H2AX regulated by phosphorylation of H2AX and ADP-ribosylation of Spt16. Mol Cell 2008; 30: 86-97.

1. A method for one or more of:
impairing or reducing the repressive function of HDAC1 and/or G9 in a eukaryotic cell; enhancing or upregulating p53 function or transcription pathway in a eukaryotic cell; and/or inhibiting the growth of a eukaryotic cell, comprising contacting the cell with a recombinant polypeptide comprising SEQ ID NO. 1 or 2 or a biological equivalent of each thereof.

2. (canceled)

3. The method of claim 1, wherein the recombinant polypeptide further comprises an agent that facilitates entry of the peptide into the cell.

4. The method of claim 3, wherein the agent that facilitates entry of the isolated or recombinant polypeptide into the cell comprises SEQ ID NO. 3 or a biological equivalent thereof.

5. The method of claim 1, wherein the recombinant polypeptide is not lysine-acetylated in a mammalian cell.

6. The method of claim 1, wherein the contacting is performed in vitro or in vivo.

7. The method of claim 1, wherein the eukaryotic cell is a cancer cell.

8. The method of claim 1, further comprising contacting the cell with an agent that inhibits the growth of the cell.

9. A method of one or ore of treating a condition mediated by p53 dysfunction and/or impaired p53 function and/or inhibiting the growth of a cell in a subject in need thereof, comprising administering to the subject an effective amount of a recombinant polypeptide comprising SEQ ID NO. 1 or 2, or a biological equivalent of each thereof.

10. (canceled)

11. The method of claim 9, wherein the recombinant polypeptide further comprises an agent that facilitates entry of the isolated or recombinant peptide into the cell.

12. The method of claim 11, wherein the agent that facilitates entry of the recombinant polypeptide into the cell comprises SEQ ID NO. 3 or a biological equivalent thereof.

13. The method of claim 9, wherein the recombinant polypeptide is not lysine-acetylated in a mammalian cell.

14. The method of claim 9, further comprising administering to the subject an effective amount of an agent that inhibits the growth of the cell in the subject.

15. The method of claim 9, wherein the p53 dysfunction or impaired p53 function is caused by the action of histone deacetylase 1 (HDAC1) and histone methyl transferase (HMT or G9a) on p53.

16. The method of claim 9, wherein the subject is suffering from cancer, wherein the cancer is linked to p53 dysfunction or impaired p53 function.

17. A recombinant polypeptide comprising SEQ ID NO. 1 or 2, a biological equivalent of each thereof.

18. The recombinant polypeptide of claim 17, wherein the polypeptide is not lysine-acetylated in a mammalian cell system.

19. The recombinant polypeptide of claim 18, wherein the polypeptide is acetylated at one or more, two or more, three or more or all four lysines and/or substituted with one, two, three or four arginines.

20. A recombinant polypeptide consisting essentially of the amino acid sequence SGRGXGGXGL GXGGAXRHRK VLRDNIQG (SEQ ID NO: 23) wherein X is independently the same or different and is one or more lysine or a side chain acetylated lysine and/or arginine.

21. The recombinant polypeptide of claim 17 or 20, wherein the recombinant polypeptide further comprises a polypeptide that facilitates entry of the polypeptide into the cell.

22. The recombinant polypeptide of claim 21, wherein the polypeptide that facilitates entry of the polypeptide into the cell comprises SEQ ID NO. 3 or a biological equivalent thereof.

23. A recombinant polynucleotide encoding the polypeptide of claim 17 or 20.

24. The recombinant polynucleotide of claim 23, further comprising regulatory polynucleotide sequences operatively linked to the polynucleotide.

25. An expression or delivery vehicle comprising the recombinant polynucleotide of claim 23.

26. An isolated host cell comprising the recombinant polypeptide of claim 17 or 20.

27. An isolated host cell comprising the recombinant polynucleotide of claim 23.

28. A composition comprising a carrier and the recombinant polypeptide of claim 17 or 20.

29. A composition comprising a carrier and the recombinant polynucleotide of claim 23.

30. A method for preparing a recombinant polypeptide comprising growing a host cell comprising the recombinant polynucleotide of claim 23 under conditions that favor the expression of the isolated or recombinant polynucleotide.

31. The method of claim 30, further comprising isolating the polypeptide from the host cell.

32. The method of claim 30, wherein the host cell is a prokaryotic cell.

33. The method of claim 32, further comprising acetylating the polypeptide at one or more lysine residues.

34. A recombinant polypeptide produced by the method of claim 30.

35. A recombinant polypeptide produced by the method of claim 33.

36. An antibody that binds an isolated or recombinant polypeptide of claim 17 or 20.

37. An isolated or recombinant polynucleotide encoding the antibody of claim 36.

38. A method for screening for an agent that inhibits or interferes with the action of histone deacetylase 1 (HDAC1) and/or histone methyl transferase (HMT or G9a) on p53 function in a eukaryotic cell, comprising contacting a cell expressing p53 and histone deacetylase 1 (HDAC1) and/or histone methyl transferase (HMT or G9a) with the agent and assaying for p53 function.

39. The method of claim 38, further comprising comparing p53 function of the cell with the ability of a recombinant polypeptide comprising SEQ ID NO. 1 or 2, a biological equivalent of each thereof, to inhibit or interfere with the action of histone deacetylase 1 (HDAC1) and/or histone methyl transferase (HMT or G9a) on p53.


SEQUENCE LISTING (23 sequences)

SEQ ID NO: 1 - protein, 28 aa, Artificial Sequence
  Description of Artificial Sequence Synthetic peptide
SGRGKGGKGLGKGGAKRHRKVLRDNIQG

SEQ ID NO: 2 - protein, 28 aa, Artificial Sequence
  Description of Artificial Sequence Synthetic peptide
SGRGRGGRGLGRGGARRHRKVLRDNIQG

SEQ ID NO: 3 - protein, 9 aa, Human immunodeficiency virus
RKKRRQRRR

SEQ ID NO: 4 - protein, 5 aa, Artificial Sequence
  Description of Artificial Sequence Synthetic peptide
KKKRK

SEQ ID NO: 5 - DNA, 21 bp, Artificial Sequence
  Description of Artificial Sequence Synthetic primer
atggaacttcgactttgtcac

SEQ ID NO: 6 - DNA, 22 bp, Artificial Sequence
  Description of Artificial Sequence Synthetic primer
aggcacaagggtacaagacagt

SEQ ID NO: 7 - DNA, 20 bp, Artificial Sequence
  Description of Artificial Sequence Synthetic primer
ccagttggaggctgaggttc

SEQ ID NO: 8 - DNA, 20 bp, Artificial Sequence
  Description of Artificial Sequence Synthetic primer
cgtttccaagggcacccatg

SEQ ID NO: 9 - DNA, 20 bp, Artificial Sequence
  Description of Artificial Sequence Synthetic primer
gtggggcgccccaggcacca

SEQ ID NO: 10 - DNA, 24 bp, Artificial Sequence
  Description of Artificial Sequence Synthetic primer
ctccttaatgtcacgcacgatttc

SEQ ID NO: 11 - DNA, 20 bp, Artificial Sequence
  Description of Artificial Sequence Synthetic primer
tggactgggcactcttgtcc

SEQ ID NO: 12 - DNA, 20 bp, Artificial Sequence
  Description of Artificial Sequence Synthetic primer
cagagtaacaggctaaggtt

SEQ ID NO: 13 - DNA, 18 bp, Artificial Sequence
  Description of Artificial Sequence Synthetic primer
gtccagcgtttgcagatg

SEQ ID NO: 14 - DNA, 18 bp, Artificial Sequence
  Description of Artificial Sequence Synthetic primer
aacgaggtgggaggagaa

SEQ ID NO: 15 - DNA, 19 bp, Artificial Sequence
  Description of Artificial Sequence Synthetic oligonucleotide
gcagatgcagagattcaac

SEQ ID NO: 16 - DNA, 25 bp, Artificial Sequence
  Description of Artificial Sequence Synthetic oligonucleotide
gacagcaagtatgaagttaaagctc

SEQ ID NO: 17 - protein, 28 aa, Artificial Sequence
  Description of Artificial Sequence Synthetic peptide
SGRGKGGKGLGKGGAKRHRKVLRDNIQG

SEQ ID NO: 18 - protein, 28 aa, Artificial Sequence
  Description of Artificial Sequence Synthetic peptide
SGRGRGGRGLGRGGARRHRKVLRDNIQG

SEQ ID NO: 19 - protein, 8 aa, Artificial Sequence
  Description of Artificial Sequence Synthetic peptide
RTLYGFGG

SEQ ID NO: 20 - protein, 37 aa, Artificial Sequence
  Description of Artificial Sequence Synthetic polypeptide
SGRGKGGKGLGKGGAKRHRKVLRDNIQGRKKRRQRRR

SEQ ID NO: 21 - protein, 37 aa, Artificial Sequence; identical to SEQ ID NO: 20
  Description of Artificial Sequence Synthetic polypeptide

SEQ ID NO: 22 - protein, 37 aa, Artificial Sequence
  Description of Artificial Sequence Synthetic polypeptide
DGAEGADVAEPFDMIEANEVKKKRKVGKRKKRRQRRR

SEQ ID NO: 23 - protein, 28 aa, Artificial Sequence
  Description of Artificial Sequence Synthetic peptide
SGRGXGGXGLGXGGAXRHRKVLRDNIQG